(19) United States
(12) Patent Application Publication
(10) Pub. No.: US 2025/0375176 A1
(43) Pub. Date: Dec. 11, 2025

(54) DATA-DRIVEN-BASED ANALYSIS OF CORONARY INDEX OF MICROCIRCULATORY RESISTANCE FROM ANGIOGRAPHY DATA

(71) Applicants: REGENTS OF THE UNIVERSITY OF MICHIGAN, Ann Arbor, MI (US); UNIVERSITY OF SOUTHERN CALIFORNIA, Los Angeles, CA (US); ANGIOINSIGHT, INC., Minnetonka, MN (US)

(72) Inventors: Carlos Alberto Figueroa-Alvarez, Ann Arbor, MI (US); Yang Zhou, Ann Arbor, MI (US); Ismael Assi, Toledo, OH (US); Haizhou Yang, Ann Arbor, MI (US); Brahmajee Kartik Nallamothu, Ann Arbor, MI (US); Krishna Garikipati, Los Angeles, CA (US); Jesse Resnick, Ann Arbor, MI (US); Himanshu Patel, Ann Arbor, MI (US); Luciano Delbono, Ann Arbor, MI (US); Domingo Uceda, Ann Arbor, MI (US)

(21) Appl. No.: 19/230,677

(22) Filed: Jun. 6, 2025

Related U.S. Application Data
(60) Provisional application No. 63/657,767, filed on Jun. 7, 2024.
(60) Provisional application No. 63/673,152, filed on Jul. 18, 2024.

(51) Int. Cl. A61B 6/50; A61B 6/00; G06T 7/11

(52) U.S. Cl. CPC A61B 6/504; A61B 6/481; G06T 7/11; G06T 2207/20081; G06T 2207/30101

(57) ABSTRACT

A method of determining microvasculature function of a vessel inspection region, the method including: obtaining angiography images of a vessel tree in the vessel inspection region, the angiography images captured over a sampling time window during which a contrast agent has been injected into the vessel tree, and applying the angiography images to a segmentation model configured to generate segmented images of the vessel tree; providing the segmented images to a contrast intensity model and, performing, by the contrast intensity model, a contrast intensity extraction on each of the segmented images to generate a contrast intensity profile of the vessel tree over the sampling time window; providing the contrast intensity profile to a microvasculature health model configured to determine a health of the microvasculature within the vessel tree based on the contrast intensity profile; and determining, using the microvasculature health model, a microvasculature health of microvasculature of the vessel tree.

### CROSS-REFERENCE TO RELATED APPLICATIONS

This application claims the priority benefit under 35 U.S.C. § 119 (e) of U.S. Provisional Application No. 63/657,767, filed Jun. 7, 2024, and U.S. Provisional Application No. 63/673,152, filed Jul. 18, 2024, which are incorporated herein by reference in their entirety.

### FIELD OF THE DISCLOSURE

The invention generally relates to determining coronary microvascular health using angiography images and a multi-stage neural network machine learning model.

### BACKGROUND

The background description provided herein is for the purpose of generally presenting the context of the disclosure. Work of the presently named inventor(s), to the extent it is described in this background section, as well as aspects of the description that may not otherwise qualify as prior art at the time of filing, are neither expressly nor impliedly admitted as prior art against the present disclosure.

Coronary Artery Disease (CAD) is among the leading causes of death in the United States, affecting more than 15 million Americans. CAD is characterized by plaque build-up from atherosclerosis in the coronary arteries, which results in the narrowing (also known as stenosis) or blockage of coronary arteries and can lead to symptoms such as angina and possibly myocardial infarction. The assessment of CAD is performed through either anatomical (e.g., localized or diffused narrowing) or functional indices, which provide a description of the ‘state of vessel occlusion’. While CAD is important to identify and characterize, the microvasculature of large vessels is additionally important to identify diseases and potential treatments for patients.

Conversely to CAD which applies to larger vessels, coronary microvascular dysfunction (CMD) or microvascular dysfunction (MVD) refers to disease in the coronary microcirculation, characterized by the loss of vasodilatory capacity and vessel rarefaction. Over 90% of the total flow resistance in the coronary system is attributed to pre-arterioles, arterioles, and capillaries in the microvasculature system. Thus, microcirculatory resistance plays a key role in regulating coronary flow, thereby balancing the equilibrium between oxygen and nutrient supply and demand. CMD is characterized by impaired blood flow and blood regulation in the microcirculation and is a critical concern in the field of cardiology. CMD encompasses a range of structural maladaptation in vessels and myocardium that disrupt the delicate balance between blood supply and demand, leading to adverse clinical outcomes, i.e. heart failure, myocardial infarction, stroke, and death.

Identifying and evaluating CMD requires extensive tests, including cardiac PET (positron emission tomography), to identify regions of the heart with deficient perfusion. Currently, there are no methods which allow quantification of CMD using angiography data. Metrics for characterizing larger vessel function and disease are often not useful and provide no details pertaining to CMD. For example, Fractional Flow Reserve (FFR) is a metric that provides a measure of the level of stenosis in an artery. However, FFR is typically reserved to determine the health of cardiovascular health of large epicardial coronary arteries, i.e., CAD, and is not suitable for assessing health in smaller vessels, i.e., microvascular disease. In short, conventional approaches for measuring and characterizing disease and vessel function for larger vessels are not applicable and cannot be used to determine CMD or microvasculature functionality.

The condition and functionality of coronary microcirculation may be physically measured by determining an index of microcirculatory resistance (IMR). The IMR is defined as the ratio between coronary pressure and the rate of flow (assessed through saline infusion) in a specific segment of a coronary artery. IMR provides a time-based approach to observing and parameterizing the functionality of flow through the microvasculature. Higher IMR values indicate greater resistance to blood flow in the microvasculature, which suggests microvascular dysfunction (e.g., presence of generalized plaque buildups in the small blood vessels of the heart) that may require attention from cardiologists.

Another metric for determining cardiovascular health is coronary flow reserve (CFR). CFR is defined as ratio of maximum (hyperemic) to baseline flow to the myocardium. Hyperemic flow is the maximum flow that a person is able to send to the heart when needed (e.g., going for a walk, a run, or playing sports). A high CFR value such as 4.0 may indicate a healthy heart of a pro-athlete, while a small CFR value such as 1.0 may indicate a diseased heart of a patient. The CFR determines the ability of a full tree (including both large and small vessels) to deliver blood to the myocardium. Reductions in CFR attributable to the large vessels can be assessed via FFR. Conversely, reductions in CFR due to CMD can be assessed via IMR.

Despite having numerous metrics, current diagnostic procedures for CAD and CMD using such metrics involves invasive and expensive techniques. Such is especially true for determining microvascular health due to the anatomical complexity of small and widespread microvasculature. Determining IMR involves complex, invasive procedures including catheterization, insertion of a pressure-sensing guidewire, etc. There are no imaging-based techniques for measuring IMR. A non-invasive imaging technique for determining microvascular health such as cardiac Position Emission Tomography (PET) is expensive, has limited availability, includes radiation exposure, requires high technical expertise, etc. Furthermore, determining thrombolysis in myocardial infraction (TIMI) grade is largely used in the setting of myocardial infarction and only offers qualitative, somewhat subjective information.

There is a need for more accurate and less-invasive techniques for diagnosis of CMD and MVD. More specifically, there is a need for less-invasive, user-independent approaches that are less expensive and more readily available for performing IMR measurements and other CMD indices with reduced risk to patients.

### SUMMARY OF THE INVENTION

In some aspects, the techniques described herein relate to a method of determining microvasculature function of a vessel inspection region, the method including: obtaining angiography images of a vessel tree in the vessel inspection region, the angiography images captured over a sampling time window during which a contrast agent has been injected into the vessel tree, and applying the angiography images to a segmentation model configured to generate segmented images of the vessel tree; providing the segmented images to a contrast intensity model and, performing, by the contrast intensity model, a contrast intensity extraction on each of the segmented images to generate a contrast intensity profile of the vessel tree over the sampling time window; providing the contrast intensity profile to a microvasculature health model configured to determine a health of the microvasculature within the vessel tree based on the contrast intensity profile; and determining, using the microvasculature health model, a microvasculature health of microvasculature of the vessel tree.

In some aspects, the techniques described herein relate to a computer-implemented method for training a microvasculature health determination system, the method including: obtaining angiography images of a plurality of vessel inspection regions from different subjects, the angiography images include subsets of angiography images captured over a full contrast agent injection cycle through corresponding vessel inspection regions, the angiography images include subsets of angiography images captured at different perspective views of corresponding vessel inspection regions; obtaining vasculature health data for each of the angiography images; performing a segmentation on each of the angiography images to generate a segmented image for each angiography image; providing the segmented images to a contrast intensity model configured perform a contrast intensity extraction on each of the segmented images to generate a contrast intensity profile of the vessel tree over a sampling time window; and providing the contrast intensity profile and the vasculature health data to a machine learning model to train the machine learning model to generate a microvasculature health of a vessel tree in a subsequently imaged vessel inspection region.

In same aspects, the techniques described herein relate to a method of assessing microvasculature function of a vessel inspection region for predicting a treatment response, the method including: obtaining angiography images of a vessel tree in the vessel inspection region, the angiography images captured over a sampling time window during which a contrast agent has been injected into the vessel tree, and applying the angiography images to a segmentation model configured to generate segmented images of the vessel tree; providing the segmented images to a contrast intensity model and, performing, by the contrast intensity model, a contrast intensity extraction on each of the segmented images to generate a contrast intensity profile of the vessel tree over the sampling time window; providing at least a portion of the contrast intensity profile to a microvasculature health model configured to predict a response of the vessel inspection region to a treatment based on a characteristic of the at least a portion of the contrast intensity profile; and generate an electronic indication of the predicted response to the treatment.

### BRIEF DESCRIPTION OF THE DRAWINGS

This patent or application file contains at least one drawing executed in color. Copies of this patent or patent application publication with color drawing(s) will be provided by the United States Patent and Trademark Office upon request and payment of the necessary fee.

The figures described below depict various aspects of the system and methods disclosed herein. It should be understood that each figure depicts an embodiment of a particular aspect of the disclosed system and methods, and that each of the figures is intended to accord with a possible embodiment thereof. Further, wherever possible, the following description refers to the reference numerals included in the following figures, in which features depicted in multiple figures are designated with consistent reference numerals.

FIG. 1 is a schematic diagram of an example system for performing anatomical and functional assessment of coronary microvascular dysfunction (CMD) and coronary artery disease (CAD).

FIG. 2A is a schematic diagram of applying a trained neural network machine learning algorithm to predict index of microcirculatory resistance (IMR), coronary flow reserve (CFR), and other indices of microvascular health as may be implemented in the system of FIG. 1, in accordance with an example.

FIG. 2B is a flow diagram of an exemplary method for determining microvascular health of a vessel tree as may be performed by the neural network model of FIG. 2A and/or machine learning framework of the system of FIG. 1.

FIG. 2C illustrates an example multi-stage neural network model for performing 3D reconstruction of vessel trees using a trained machine learning (ML) model.

FIG. 2D shows a process for performing 3D reconstruction of coronary vessel trees using a trained ML model.

FIG. 2E illustrates an example of both a high-fidelity, or 3D vessel tree, representation and a low-fidelity, or 1D vessel tree, representation of a vessel tree.

FIG. 2F is a schematic diagram of an example implemented architecture of a neural network with angiogram inputs and output 3D reconstructed vessel tree.

FIG. 3A is a schematic diagram of training a neural network machine learning algorithm as may be implemented in the system of FIG. 1, in accordance with an example.

FIG. 3B is a schematic diagram of an example system pathway for utilizing 2D images to generate contrast intensity profiles for determining microvascular health using machine learning models.

FIG. 3C is a schematic diagram of an example system pathway for utilizing 3D images to generate contrast intensity profiles for determining microvascular health using machine learning models.

FIG. 4 shows angiography images, segmented images, and a contrast intensity profile for determining microvascular health according to examples.

FIG. 5 shows data from a multi-physics model simulating contrast injection, generated angiography images, segmented images, and a resulting contrast intensity profile for the generated 3D multi-physics models.

FIG. 6A shows a multi-physics model of contrast injection, with lumped parameter models (LPM) that are used as boundary conditions for the hemodynamic simulations, and a catheter to simulate contrast injection.

FIG. 6B provides a step function for simulated contrast injection and Navier-stokes and convection-diffusion equations within 3D coronary artery for calculation flow dynamics and transport of contrast.

FIG. 6C provides illustrations of simulated hemodynamics and transport of contrast at specified times for pressure, velocity, and iodine contrast using the multi-physics computational model and equations of FIGS. 6A-6B.

FIG. 7A illustrates generated contrast intensity profiles from angiography images generated with the multi-physics model of contrast injection (FIGS. 6A-6C) with set values of microvascular resistance.

FIG. 7B illustrates examples of contrast intensity profiles generated via contrast intensity module. Curves are separated between increasing/rising and decreasing/falling slopes and are generated using clinical angiography images.

FIG. 7C illustrates contrast intensity profiles generated for individual coronary arteries: left anterior descending (LAD) and obtuse marginal (OM), depicting contrast uptake and washout for those individual vessels.

FIG. 7D depicts examples of two contrast intensity profiles for two different coronary vessels in the same patient, a contrast intensity profile for the obtuse marginal (OM) and another for the right posterior descending artery (RPDA), indicating the downslope in the OM profile is steeper than in the RPDA profile (blue lines). FIG. 7D further illustrates a table showing statistically significant differences between the downslope of patent (−5425) vs occluded (−4009) bypasses in different coronary vessels, demonstrating a strong indication that features in the CIP are predictive of the state of microvascular health.

FIG. 7E is a plot and table illustrative of a correlation between the ground truth multi-physics model IMR using a testing dataset generated according to the model and equations of FIGS. 6A-6C (x-axis) and predicted IMR using a ML model (y-axis)

FIG. 7F is a plot and table illustrative of a correlation between ground truth multi-physics model CFR using a testing dataset generated according to the model and equations of FIGS. 6A-6C (x-axis) and predicted CFR using a ML model (y-axis).

FIG. 8A represents plots of pre-hyperemia state contrast intensity profiles taken at different perspectives of a vessel tree for 3 different subjects with different values of IMR.

FIG. 8B provides plots of increasing slopes of contrast profiles taken of pre-hyperemia state at different perspectives of a vessel tree.

FIG. 8C provides plots of decreasing slopes of contrast profiles taken of pre-hyperemia state at different perspectives of a vessel tree.

FIG. 8D provides plots of contrast intensity profiles for a microvascular measurement of a post-hyperemia state of a vessel tree.

FIG. 8E are plots providing direct comparisons of pre-hyperemia (baseline) state and post-hyperemia state of contrast intensity profiles measured at different perspectives of a vessel tree for three different patients.

FIG. 9A presents a flow diagram of both machine learning training framework 902 and trained machine learning framework for determine microvascular health parameters from 2D and 3D images as described herein.

FIG. 9B provides a block diagram of a detailed depiction of an example microvascular health machine learning model in inference mode, to predict IMR from a single contrast intensity profile input.

FIG. 9C provides a block diagram of a detailed depiction of another example of microvascular health machine learning model in inference mode, to predict CFR from multiple contrast intensity profile inputs.

FIG. 10A shows a process to create contrast intensity profiles from angiographic data, including normalized and smoothed curves, using a method for automatic frame count requiring the definition of minimum and maximum thresholds for contrast intensity.

FIG. 10B illustrates results for automatic frame count corresponding to rising slopes (filling) in a series of patients using clinical angiographic data.

FIG. 10C illustrates correlation between clinical TIMI frame counts and automatic frame count for a series of patients.

FIG. 10D illustrate comparison between filling (rising) slopes and emptying (falling) slopes and TIMI frame counts.

### DETAILED DESCRIPTION

Coronary microvascular dysfunction (CMD) is characterized by impaired blood flow and blood regulation in the microcirculation and is a critical concern in the field of cardiology. CMD encompasses a range of structural maladaptation in vessels and myocardium that disrupt the delicate balance between blood supply and demand, leading to adverse clinical outcomes. The index of microcirculatory resistance (IMR) is regarded as one of the current gold standards for evaluating coronary microcirculatory function. IMR has reliably predicted myocardial viability after primary angioplasty following myocardial infarction as well as the extent and severity of myocardial infarction in patients. Another useful index to characterize coronary microvascular function is the coronary flow reserve (CFR), the ratio of maximum (hyperemic) to baseline flow in a given vessel or tree. However, IMR and CFR assessment remains underused in many medical settings due to its invasive nature as it requires additional placement of a coronary wire in the vessel. Therefore, a non-invasive, data-driven approach for IMR and CFR assessment is highly desired. A data-driven framework (microvasculature health model) capable of harnessing information encapsulated within angiography data, the most commonly used modality for coronary artery disease assessment, with a multi-physics computation model to quantify IMR and CFR, and other microvascular health metrics, can be developed. Additionally, the described systems and methods may be used to calculate microvascular resistance reserve (MRR), an index specifically related to the performance of microvasculature in the ability to increase blood and oxygen flow, by examining changes from a baseline to a hyperemic condition.

Provided are techniques for assessing the microvascular health of a vessel tree within a vessel inspection region. Multiple angiography images of the vessel inspection region are captured over one or more sampling time windows following the injection of a contrast agent into the vessel tree. These images may then be binarized using a segmentation model. A contrast intensity profile of the vessel tree or of a single branch in the vessel tree is then generated over the sampling period. A microvascular health model uses this contrast intensity profile to evaluate the microvascular health (e.g., by determining IMR, CFR, MRR or other microvascular health values) of the vessels in the inspection region.

In some examples, a multi-physics model can be developed and calibrated to develop pairs of generated angiography images and known coronary microvascular resistance values to train the microvascular health model. The multi-physics model may be a multi-physics computational fluid dynamics (CFD) model of contrast injection. The microvascular health model can be trained to interpret the multi-physics model generated angiography data paired with the known coronary microvascular resistance values, i.e. the dynamics of the contrast injection and washout within the coronary arteries, and study the correlation between these dynamics and IMR, CFR, or MRR if two hemodynamic conditions (baseline and hyperemic states) are generated.

Microvascular health models herein may be trained on various types of input images. For example, techniques herein may be implemented using three-dimensional (3D) images of vessel inspection regions and/or 3D models of such vessel inspection regions. These 3D images and models may be volumetric images and models, for example. In various examples, techniques herein may be implemented using two-dimensional (2D) angiography images, collected either natively or derived from one or more perspectives of the 3D models of vessel inspection regions. For example, 3D images or models can be constructed using 2D angiography images, and the 3D representations can then be used to determine IMR and other metrics as described herein. Additionally, techniques herein may be implemented using multiple 2D angiography images of various perspectives to determine IMR and other metrics.

Thus, in various examples, the microvasculature health model may be trained using 3D images and models or on 2D images and models and additionally on synthetically generated data or on clinically obtained data. A microvasculature health model, combined with a contrast intensity model that deals with 3D segmentation data, allows for accurate assessment of fluid dynamics and for the derivation of metrics indicative of CMD (e.g., CFR, MRR and IMR) due to the ability to observe and consider all three-directions of fluid flow.

It should be understood that the term “3D image” and “3D model” may be used herein to described data pertaining to 3D representations of various elements such as vessel trees, vessels, coronary regions and tissues, etc. The 3D images and models may include data representation of single vessels or vessel trees, blood flow, and contrast dynamics, in a three-dimensional coordinate system. The 3D data of the 3D images may be generated, analyzed and used for training machine learning models in a manner that is independent of an orientation or perspective of the 3D image or model. As such, the 3D data allows for volumetric understanding and analysis for generating IMR, CFR, and other fluid dynamics related to coronary function, vessel health, and CMD. In any examples, the 3D images may include voxels that are indicative of graphical information, hemodynamics information, or other data and information as may be used for performing the methods described herein.

### System Overview

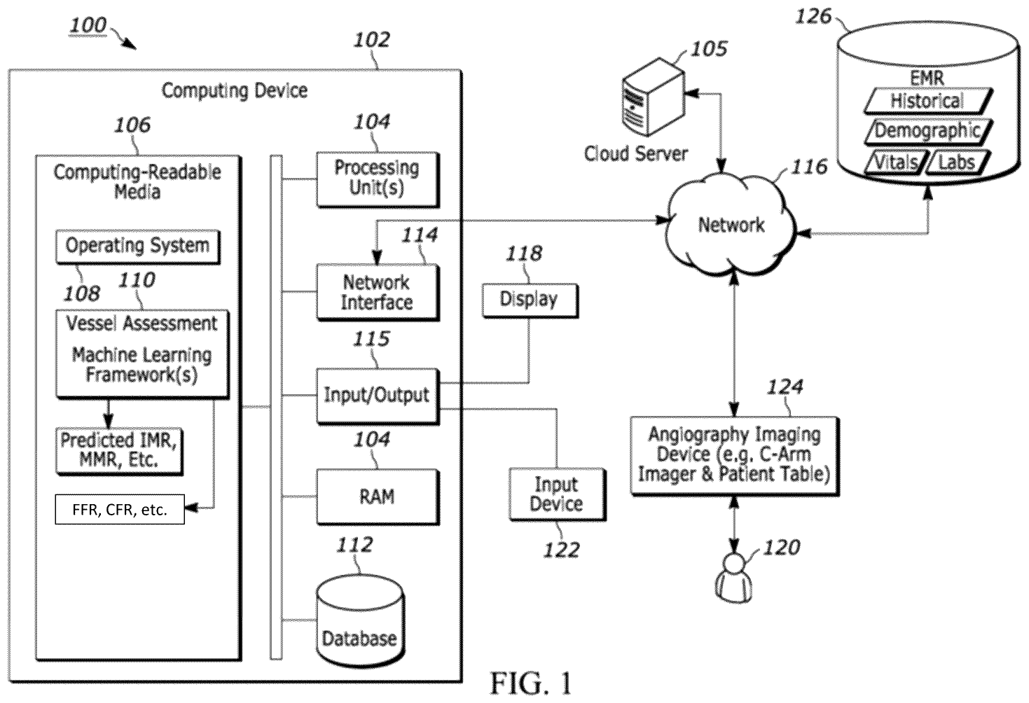

FIG. 1 illustrates a vessel assessment system 100 that may be used (e.g., in an inference mode) to assess microvasculature health of a vessel region in isolation or in combination with assessing coronary health more broadly and that may be used in train a machine learning model (e.g., in a training mode) for affecting such assessments. In the illustrated example, the vessel assessment system 100 includes a computing device 102 “or “signal processor” or “diagnostic device”) configured to collect angiography image data from a patient 120 via an angiography imaging device 124 in accordance with example techniques executing the functions of the disclosed embodiments. In examples, the angiography imaging device 124 may include one or more devices capable of obtaining 2D images of vessels or regions of vessels. As additionally described further herein, the system 100 may further generate 3D images from 2D images of vessels or regions of vessels to perform the methods on 3D image data rather than 2D angiography images. The vessel assessment system may be used to implement the training and implementation of the machine learning models and machine learning frameworks 110 for determining microvasculature health of a vessel inspection region described herein. A “vessel inspection region” for instance can comprise a single vessel, or multiple vessels, within a tree, and the analysis can be applied independently thereto.

As illustrated, the system 100 may be implemented on the computing device 102 and in particular on one or more processing units 104, which may represent Central Processing Units (CPUs), and/or on one or more or Graphical Processing Units (GPUs), including clusters of CPUs and/or GPUs, any of which may be cloud based. Features and functions described for the system 100 may be stored on and implemented from one or more non-transitory computer-readable media 106 of the computing device 102. The computer-readable media 106 may include, for example, an operating system 108 and a CAD machine learning (deep learning and/or neural networks) framework 110 having elements corresponding to that of deep learning framework described herein. More generally, the computer-readable media 106 may store trained deep learning models, including vessel segmentation machine learning models, flow extraction machine learning models, microvasculature health machine learning model, Graph-theory or other neural network based reduced order models, executable code, etc. used for implementing the techniques herein. Additionally, the computer-readable media 106 may store executable instructions for training a machine learning (ML) model such as a deep learning model, and/or neural network model as described herein. The computer-readable media 106 and the processing units 104 may store image data, segmentation models or rules, fluid dynamic classifiers, data sets indicative of 3D reconstructions of vessel trees, synthetic vessel trees, and other data herein in one or more databases 112. As discussed in examples herein, the vessel assessment machine learning framework 110 applying the techniques and processes herein (e.g., various different neural networks) may determine predicted IMR values, FFR, CFR, MRR, and other fluid dynamic assessments, state of vessel occlusion data (such as degree of stenosis), and/or microvascular disease data.

The computing device 102 includes a network interface 114 communicatively coupled to the network 116, for communicating to and/or from a portable personal computer, smart phone, electronic document, tablet, and/or desktop personal computer, or other computing devices. The computing device further includes an I/O interface 115 connected to devices, such as digital displays 118, user input devices 122, etc. As described herein, the computing device 102 generates indications of vascular health for a subject, which may include states of vessels in the vasculature, such as CAD or other state of vessel occlusion (anatomical and functional through an FFR calculation, through an iFR calculation, or through a QFR calculation), and which may include states of microvascular disease prediction (by contrasting changes in distal resistance when two hemodynamic states are recorded, estimating IMR, CFR, MRR or other index of CMD), as an electronic document that can be accessed and/or shared on the network 116.

In the illustrated example, the computing device 102 is communicatively coupled, through the network 116, to an electronic medical records (EMR) database 126. The EMR database 126 may be a network accessible database or dedicated processing system. In some examples, the EMR database 126 includes data on one or more respective patients. That EMR data may include vital signs data (e.g., pulse oximetry derived hemoglobin oxygen saturation, heart rate, blood pressure, respiratory rate), lab data such as complete blood counts (e.g., mean platelet volume, hematocrit, hemoglobin, mean corpuscular hemoglobin, mean corpuscular hemoglobin concentration, mean corpuscular hemoglobin volume, white blood cell count, platelets, red blood cell count, and red cell distribution width), lab data such as basic metabolic panel (e.g., blood urea nitrogen, potassium, sodium, glucose, chloride, CO2, calcium, creatinine), demographic data (e.g., age, weight, race and gender, zip code), less common lab data (e.g., bilirubin, partial thromboplastin time, international normalized ratio, lactate, magnesium and phosphorous), and any other suitable patient indicators now existing or later developed (e.g., use of O₂, Glasgow Coma Score or components thereof, and urine output over past 24 hours, antibiotic administration, blood transfusion, fluid administration, etc.); and calculated values including shock index and mean arterial pressure. The EMR data may additionally or alternatively include chronic medical and/or surgical conditions. The EMR data may include historical data collected from previous examinations of the patient, including historical FFR, iFR, IMR, CFR, MRR, or QFR data. Determinations of stenosis, vascular disease prediction, vascular resistance, CFD simulation data, and other data will be produced in accordance with the techniques herein. The EMR database 126 may be updated as new data is collected from the angiography imaging device 124 and assessed using the computing device 102. In some examples, the techniques may provide continuous training of the EMR database 126. Additionally, the EMR may store three-dimensional images and models of vessels, organs, or vessel inspection regions as further described herein.

In conventional angiography imaging applications, angiography images are captured by the medical imager and then sent to an EMR for storage and further processing, including, in some examples image processing, before those images are sent to a medical professional. With the present techniques, the state of occlusion, stenosis, and state of microvascular disease can be determined at computing device based on the angiography images, and without first offloading those images to the EMR database 126 for processing. In total, the techniques proposed herein are able to reduce analysis times for cardiologists considerably, and, in part, due to this bypassing of the EMR database 126 for processing. The EMR database 126 may be simply poled for data during analysis by the computing device 102 and used for storage of state determinations and other computations generated by the techniques herein. Indeed, there are numerous benefits that result from the faster and more automated analyses resulting from the present techniques. For example, determining IMR or CFR values using one or more of machine learning frameworks from vessel assessment machine learning frameworks 110 can output predicted IMR or CFR values for a patient, in which the cardiologists can subsequently use it to determine microvascular health of a patient.

In the illustrated example, the system 100 is implemented on a single server. However, the functions of the system 100 may be implemented across distributed devices connected to one another through a communication link. In other examples, functionality of the system 100 may be distributed across any number of devices, including the portable personal computer, smart phone, electronic document, tablet, and desktop personal computer devices shown. In other examples, the functions of the system 100 may be cloud based, such as, for example one or more connected cloud CPU(s) or computing systems, labeled 105, customized to perform machine learning processes and computational techniques herein. The network 116 may be a public network such as the Internet, private network such as research institution's or corporation's private network, or any combination thereof. Networks can include, local area network (LAN), wide area network (WAN), cellular, satellite, or other network infrastructure, whether wireless or wired. The network can utilize communications protocols, including packet-based and/or datagram-based protocols such as internet protocol (IP), transmission control protocol (TCP), user datagram protocol (UDP), or other types of protocols. Moreover, the network 116 can include a number of devices that facilitate network communications and/or form a hardware basis for the networks, such as switches, routers, gateways, access points (such as a wireless access point as shown), firewalls, base stations, repeaters, backbone devices, etc.

The computer-readable media 106 may include executable computer-readable code stored thereon for programming a computer (e.g., comprising a processor(s) and GPU(s)) to the techniques herein. Examples of such computer-readable storage media include a hard disk, a CD-ROM, digital versatile disks (DVDs), an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory) and a Flash memory. More generally, the processing units of the computing device 102 may represent a CPU-type processing unit, a GPU-type processing unit, a field-programmable gate array (FPGA), another class of digital signal processor (DSP), or other hardware logic components that can be driven by a CPU.

It is noted that while example deep learning frameworks and neural networks herein are described as configured with example machine learning architectures, any number of suitable convolutional neural network architectures may be used. Broadly speaking, the deep learning frameworks herein may implement any suitable statistical model (e.g., a neural network or other model implemented through a machine learning process) that will be applied to each of the received images.

In some examples, when a statistical model is implemented using a neural network, the neural network may be configured in a variety of ways. In some examples, the neural network may be a deep neural network and/or a convolutional neural network. In some examples, the neural network can be a distributed and scalable neural network. The neural network may be customized in a variety of manners, including providing a specific top layer such as but not limited to a logistics regression top layer. A convolutional neural network can be considered as a neural network that contains sets of nodes with tied parameters. A deep convolutional neural network can be considered as having a stacked structure with a plurality of layers. The neural network or other machine learning processes may include many different sizes, numbers of layers and levels of connectedness. Some layers can correspond to stacked convolutional layers (optionally followed by contrast normalization and max-pooling) followed by one or more fully-connected layers. The present techniques may be implemented such that machine learning training may be performed using a small dataset, for example less than 20,000 images, 15,000 images, 10,000 images, less than 1,000 images, or less than 500 images. In an example, approximately 15,00 images were used (three images per vessel tree with 5,000 vessel trees).

### Determining Microvascular Health via Machine Learning Model and Contrast Image Profiles

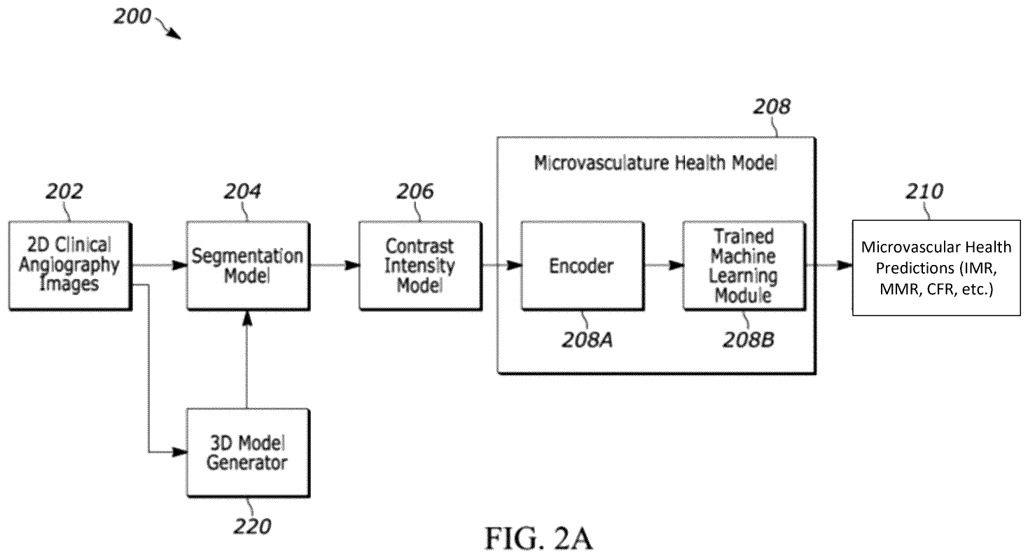

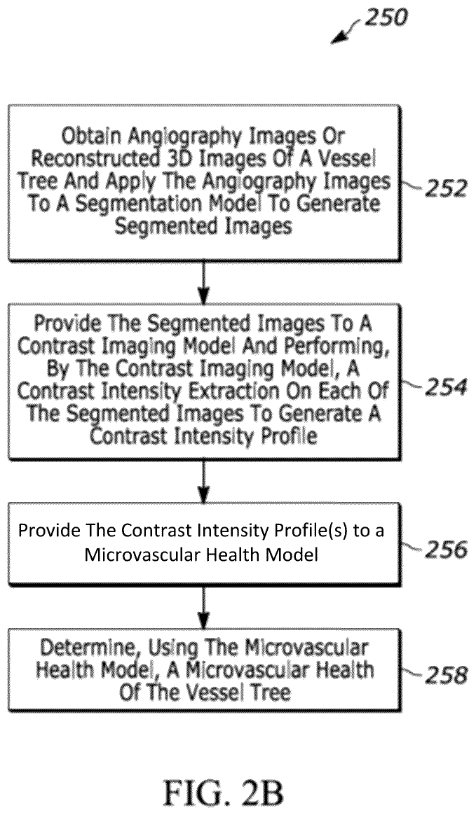

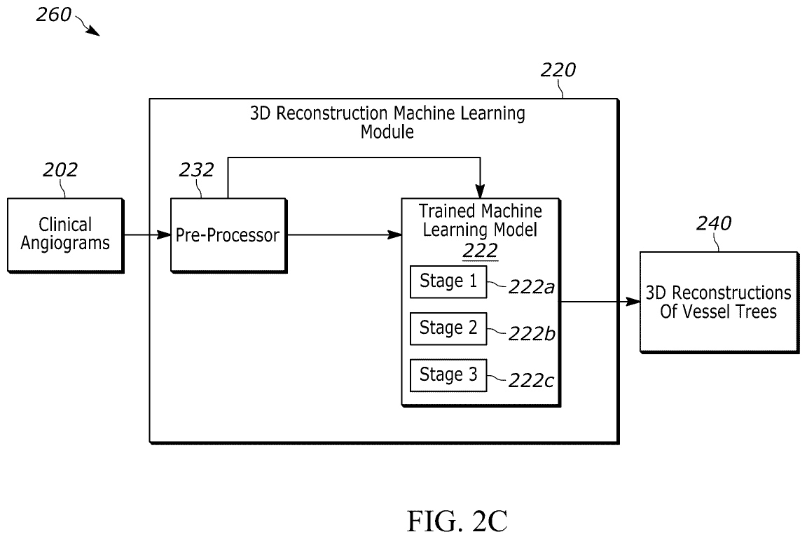

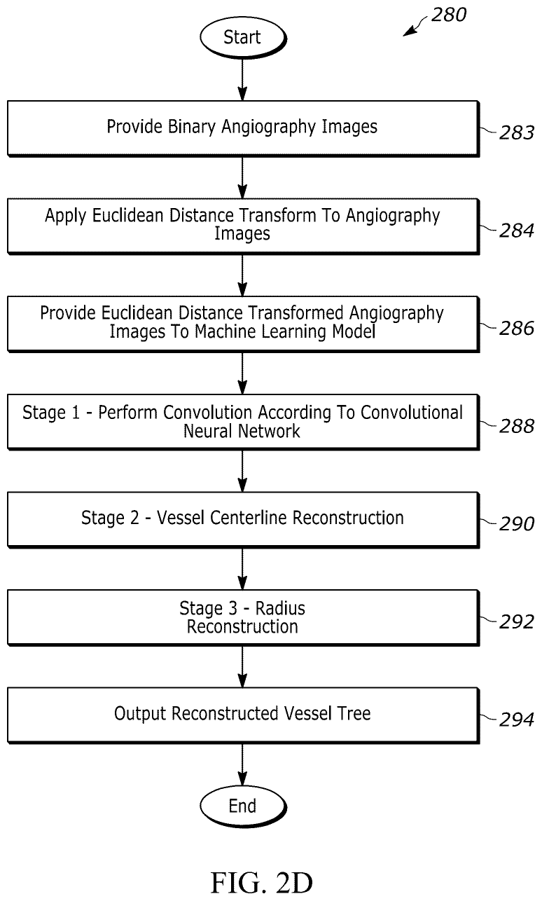

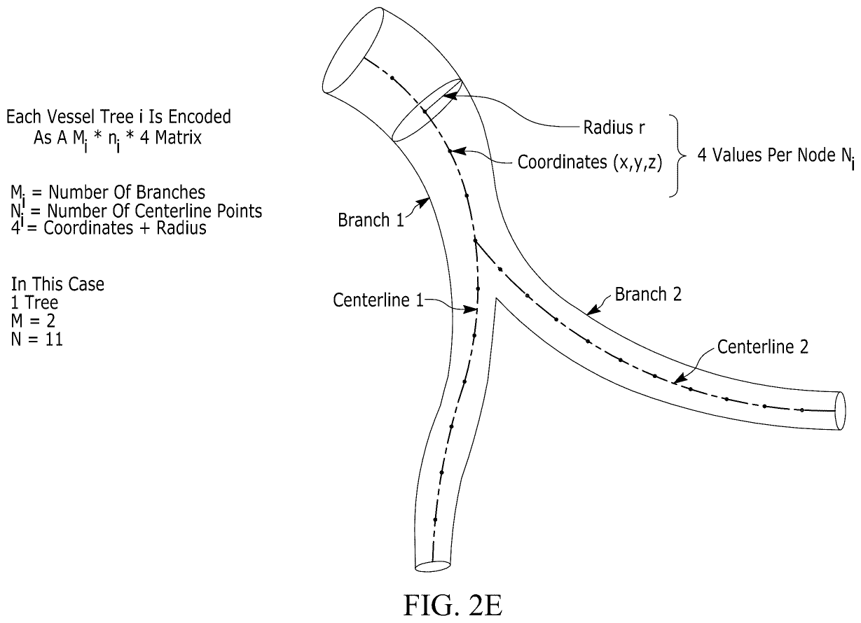

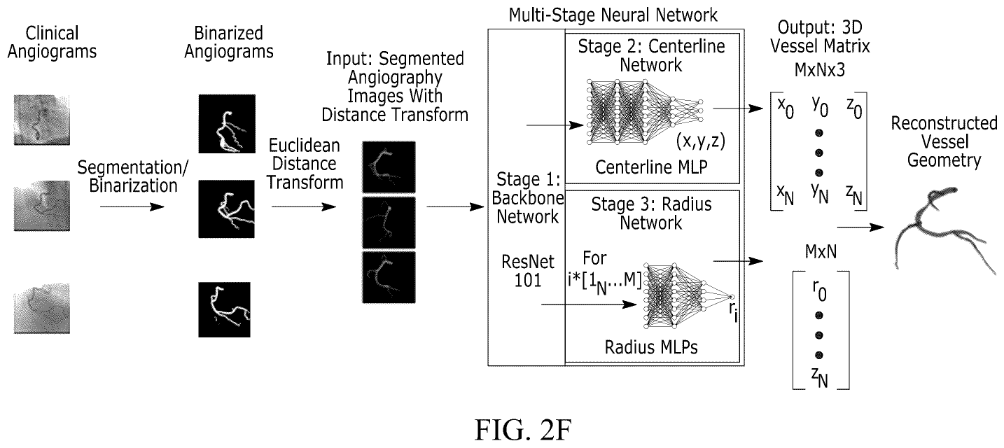

FIG. 2A illustrates an example machine learning model 200 for determining microvasculature health of a vessel tree or a single vessel in a vessel inspection region. FIG. 2B is a flow diagram of an exemplary method for determining microvascular health of a vessel tree as may be performed by the machine learning model 200 and/or system 100. At block 252, clinical angiography images 202 are obtained of a target vessel region. The plurality of clinical angiography images 202 may be input to the model 200 of FIG. 2A.

The plurality of clinical angiography images 202 may be angiography images of the vessel tree in the vessel inspection region over a sampling time window during which a contrast agent (e.g., iodine dye) has been injected into the vessel tree. The plurality of angiography images 202 capture time-series imagery of the contrast agent flowing through the vessel tree region. Each clinical angiography image of the plurality of clinical angiography images 202 may represent a certain time in the sampling time window. For example, one clinical angiography image may represent an image taken at a time of 0.5 sec of the sampling time window while another clinical angiography image may represent an image or frame taken at 1 sec of the sampling time window. As such, each image or frame is obtained at a different time of the sampling time window.

The plurality of clinical angiography images 202 may be angiography images of the vessel tree in the vessel inspection region captured at a certain perspective. For example, the plurality of clinical angiography images 202 may represent angiography images taken from a patient for a front view of the microvasculature. Different pluralities of clinical angiography images 202 may be taken at different perspectives of a same vessel tree.

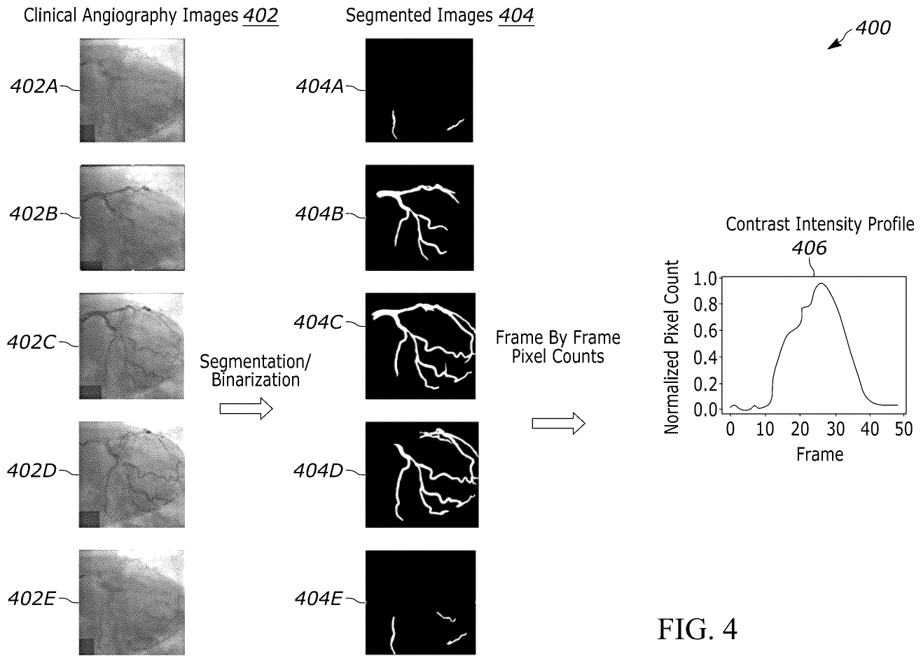

The segmentation model 204 then performs segmentation on the input angiography images 202 to generate a plurality of binarized segmented images at block 254. The binarized segmented angiography images increase the image contrast of the contrast agent in the vessel tree for the images as the contrast agent enters, and further extraction of the contrast agent as it passes through the vessel tree. The segmentation model 204 provides the binarized segmented images to a contrast intensity model 206. Examples of binarized segmented images are further provided and described in FIG. 4.

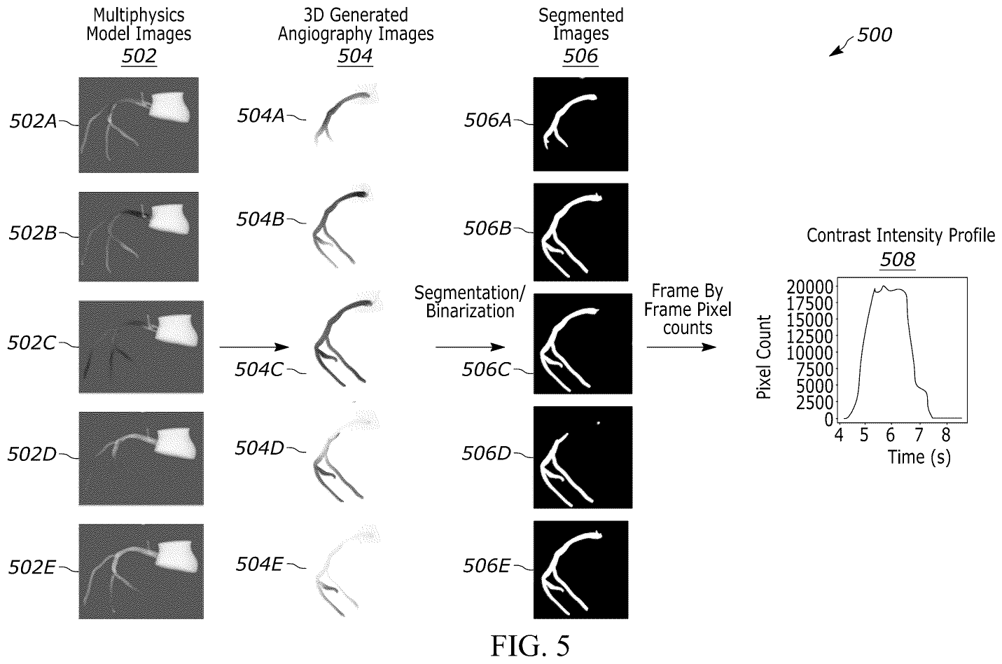

The contrast intensity model 206 determines then performs a contrast intensity extraction on each of the segmented images to generate a contrast intensity profile of the vessel tree over the sampling time window, at block 254. The contrast intensity profile is a measure of the number of pixels indicative of contrast agent present in an angiography. Example contrast intensity profiles are further described in reference to FIGS. 4 and 5. The contrast intensity profile may be a two-dimension x-y plot, with the x-axis being sampling time window and y-axis being the number of illuminated pixels or voxels in a segmented image. While in various illustrated examples, the segmented images are binarized, which means that the illuminated pixels appear as “white” pixels, a person of ordinary skill in the art would recognize that the pixels being used to generate contrast intensity profiles may be of any intensity above a predetermined threshold above non-illumination (e.g., any intensity above a predetermined low intensity value in grayscale images) and the pixels may appear as any color in the image as long as the pixels are distinct to indicate regions of a vessel tree having a contrast flowing through the vessel tree or vessel.

The contrast intensity profiles provide curves that plot the increase of contrast agent into a vessel tree, and the reduction of contrast agent over time across a set of time-series angiography images. The contrast intensity profile includes several features such as rising slope, falling slope, peak width, etc. that may be associated with microvascular health or disease, and may be indicative of IMR values as described herein. In any examples, the contrast image profiles herein (including contrast intensity profiles, etc.) may include data collected during a hyperemic flow state and/or a resting flow state. The contrast intensity model 206 then provides the contrast intensity profile(s), and or data indicative of the contrast intensity profile(s), to the microvasculature health model 212, at block 256.

The microvasculature health model 208 determines microvasculature health of the vessel tree from the contrast intensity profile at block 258. The microvasculature health model 208 is trained using contrast intensity profiles and associated microvasculature health parameters and metrics to determine microvasculature health metrics from input contrast image profiles. For example, a contrast intensity profile may be provided to the microvasculature health model 208, and the microvasculature health model 208 may output IMR, CFR or other CMD indices 210 based on various features of the contrast intensity profile (e.g., a rising slope, falling slope, length of time of entire curve, maximum duration, total area under the curve, and similarly for normalized CIPs (maximum value of the curve is set to one), etc.).

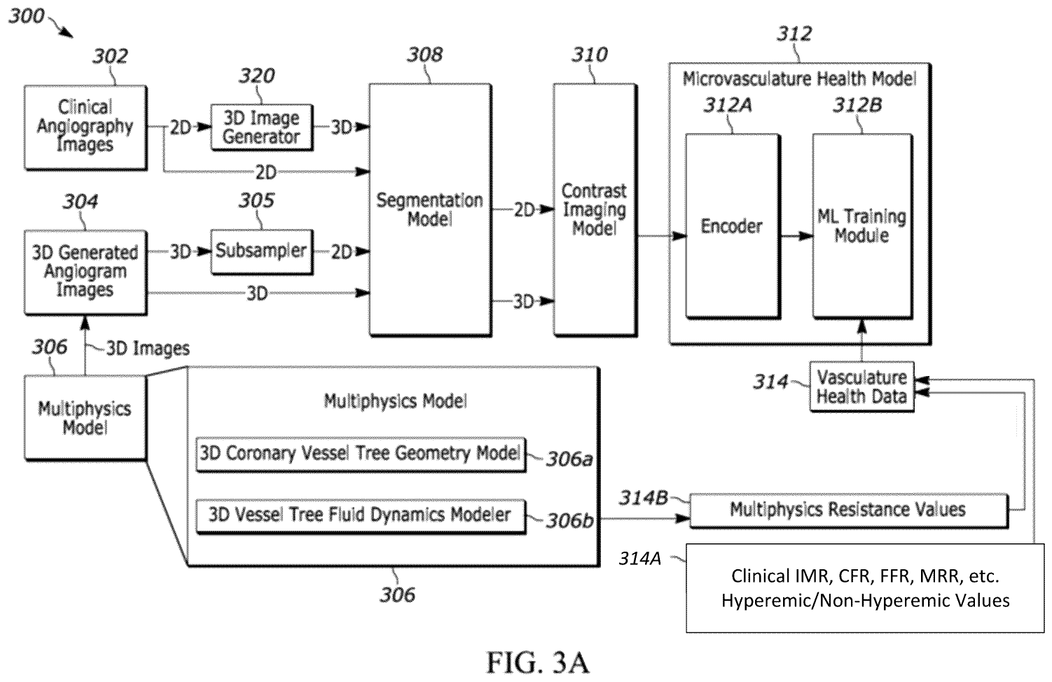

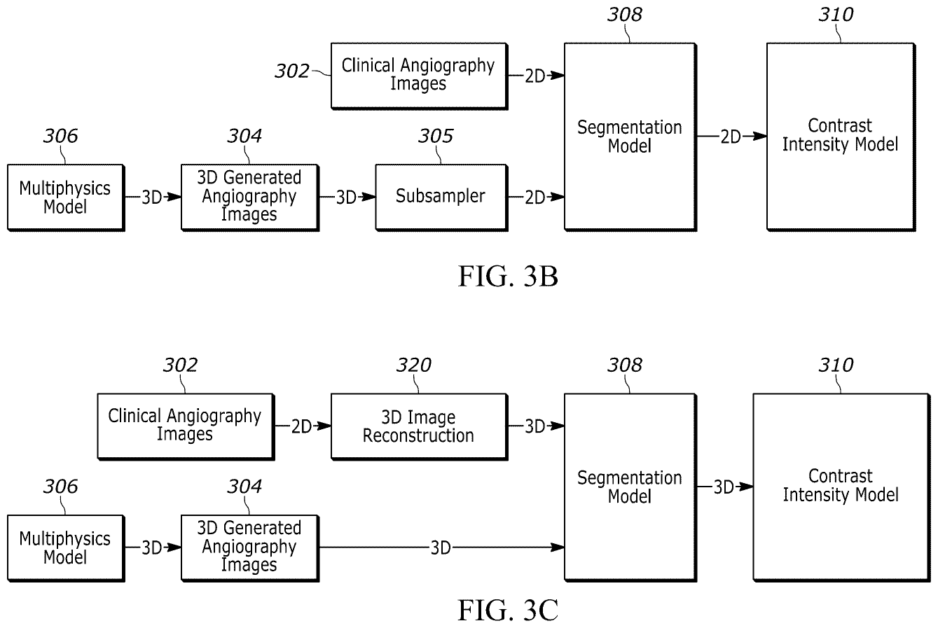

The microvasculature health model 208 includes an encoder 208A and a ML trained module 208B. The ML trained module 208 may be a machine learning model that had been trained as described in accordance with FIG. 3A. The microvasculature health model 208 receives the contrast intensity profile(s) from the contrast intensity model 206, and the encoder 208A may reduce the contrast intensity profile to a lower dimension and determine feature maps of the contrast intensity profile. The ML trained module 208B may then use the features maps extracted from the contrast intensity profile to determine a microvasculature health prediction 210 (e.g., IMR, CFR, MRR, etc.) of the vessel tree. It will be appreciated the techniques described herein in reference to a vessel tree may be performed for a single vessel, such as, for example generating a contrast intensity profile for a single branch rather than for all the vessels in a captured image.

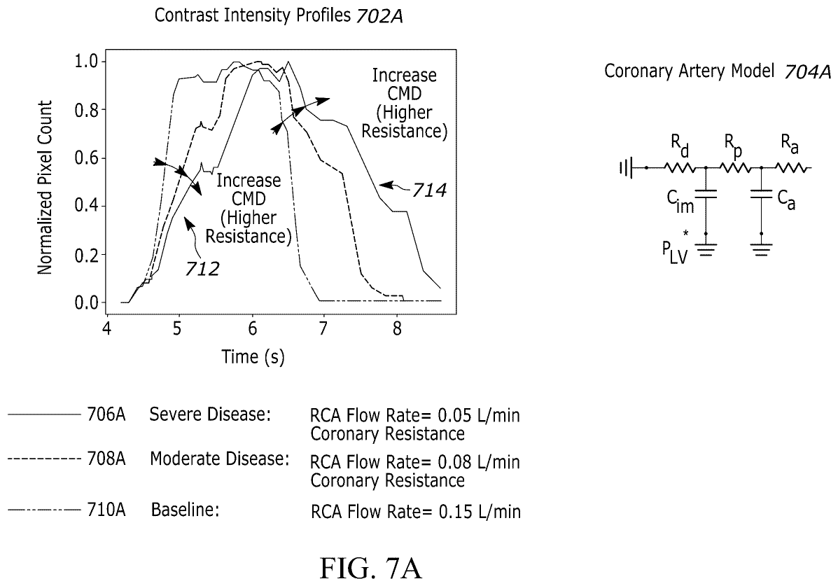

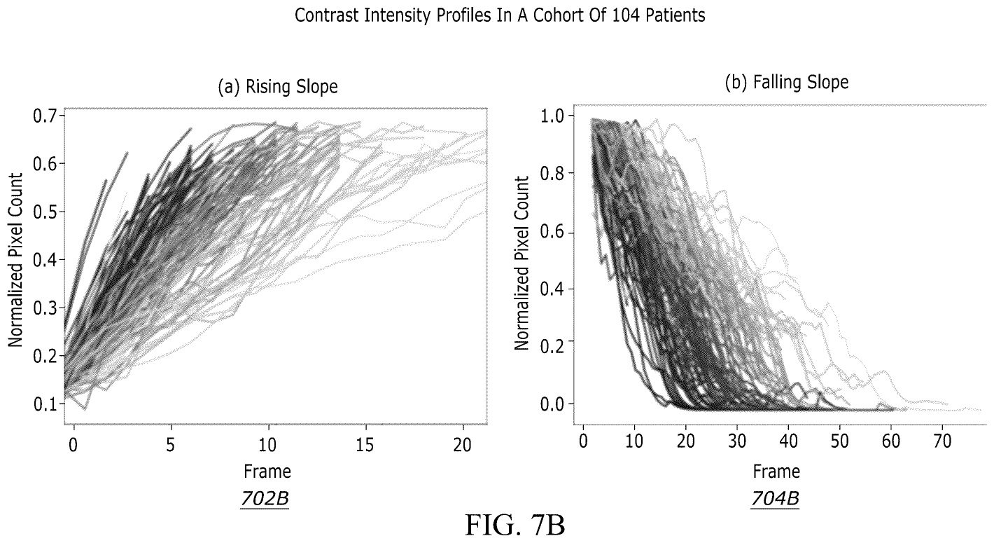

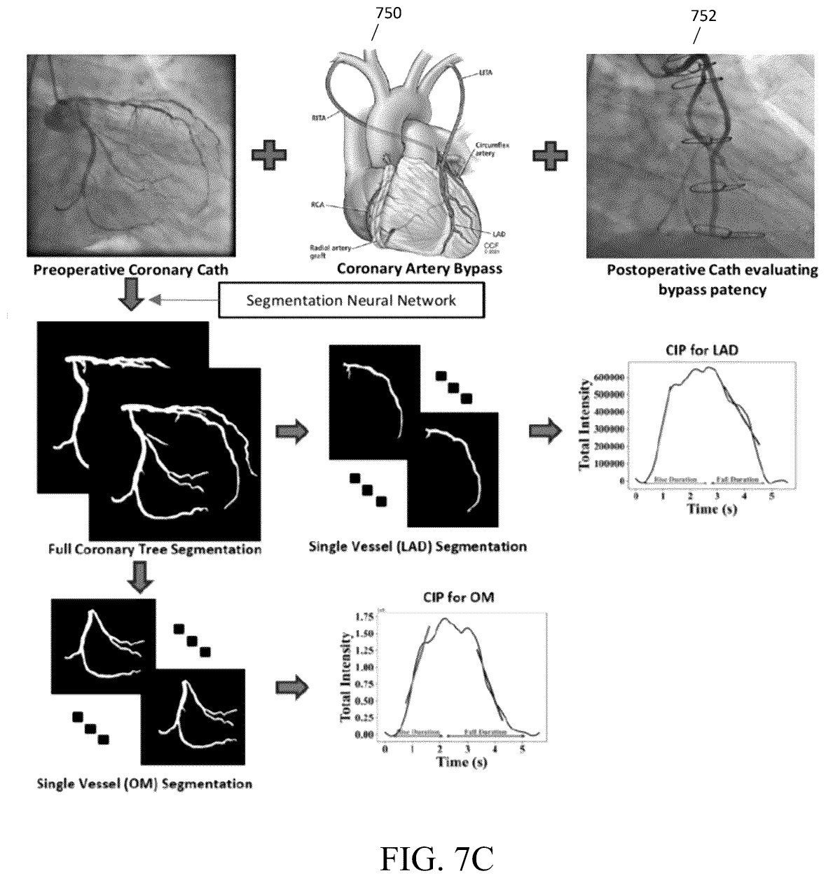

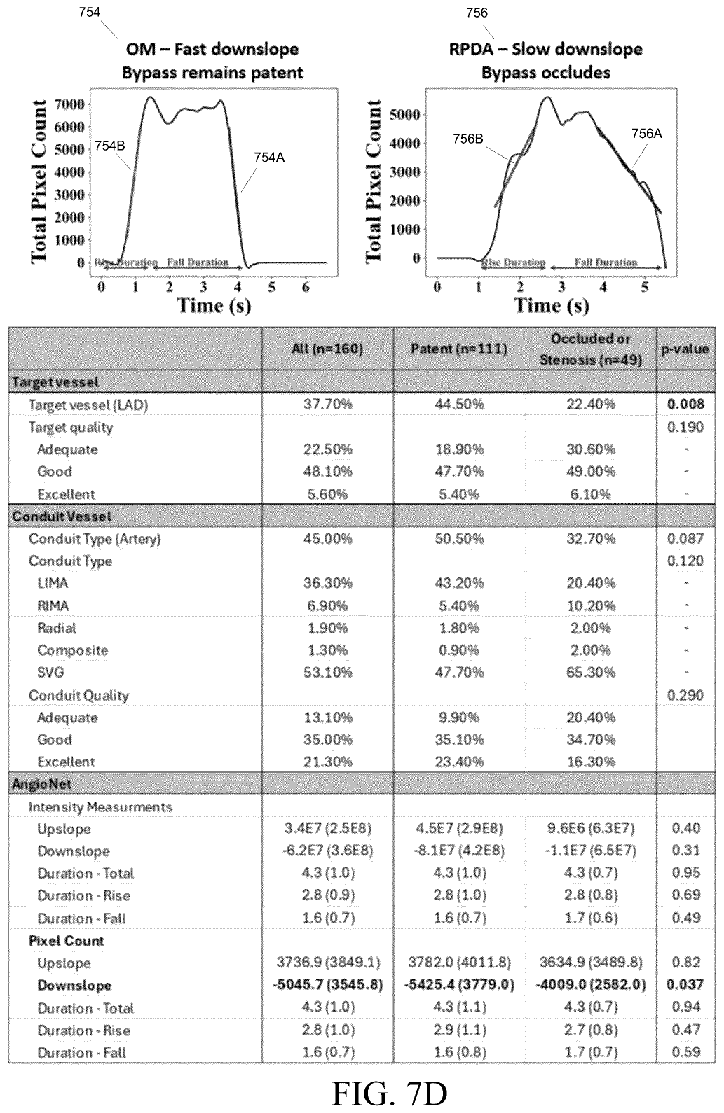

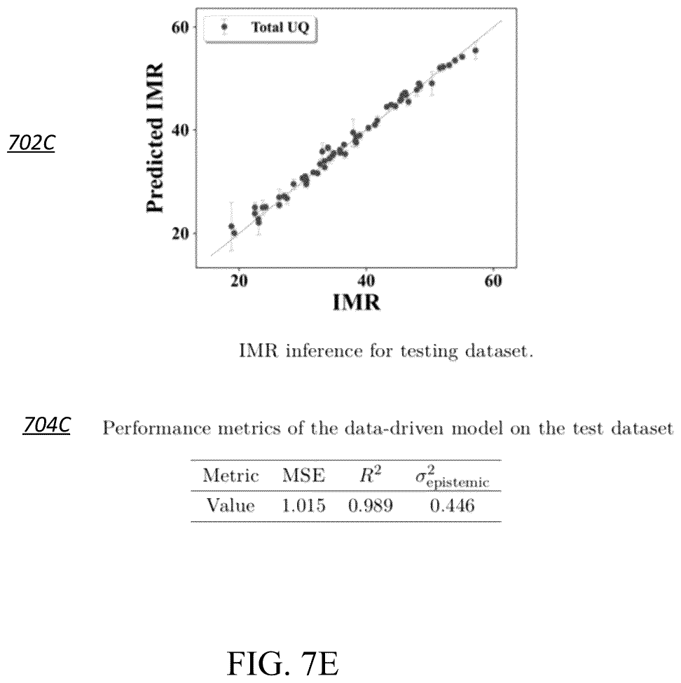

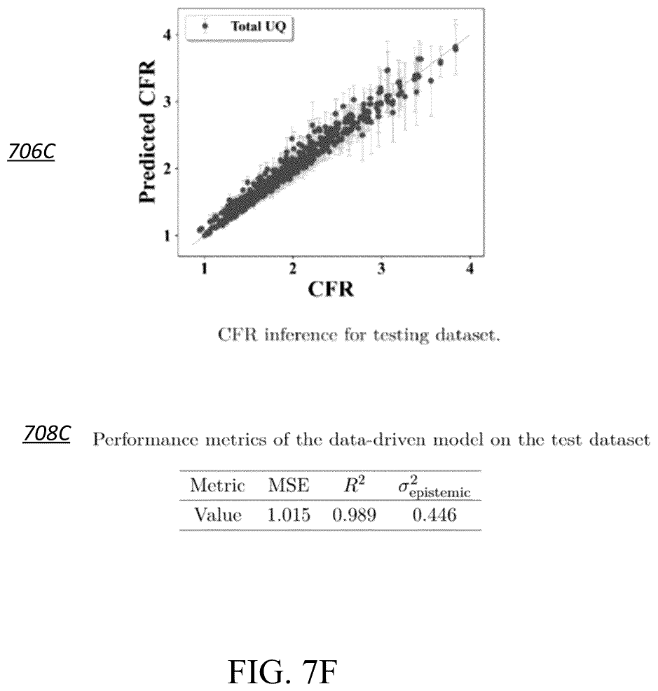

In some embodiments, a plurality of generated angiography images (e.g., via a multi-physics model and vessel tree geometries) can be used to determine microvascular health predictions 210. Determining microvascular health predictions 210 from generated images and data sets allows for the comparison and accuracy testing for the microvasculature health model 208, including for example that of the ML trained model 208B therein. An example of performing such comparison and testing may be found in reference to FIGS. 7E and 7F.

### 3D Image Construction and Microvascular Health Determination

In some embodiments, clinical angiography images 202 of different perspectives but a same vessel tree may be provided to a 3D model generator 220. The 3D model generator may be a trained ML model to construct 3D images and 3D models of a vessel tree from input angiography images. For example, the pluralities of clinical angiography images 202 may be combined by the 3D model generator 220 to create a time-series of 3D images of a vessel region having contrast agent injected and flow through the vessel tree. The constructed 3D images may then be provided to the segmentation model 204 (block 252 of FIG. 2B), and segmented images may further be provided to the contrast intensity model 206 to determine contrast intensity profiles at block 254 of FIG. 2B. In the case of 3D images, the contrast intensity model 206 may determine a number of voxels of contrast in a given 3D image to generate a contrast intensity profile. The microvascular health model 208 then receives the contrast intensity profiles (block 256) and further determines microvascular health parameters (e.g., IMR, CFR, MRR, etc.) from the contrast intensity profiles of the 3D images at block 258.

FIG. 2C illustrates an example multi-stage neural network model 200 for performing 3D reconstruction of vessel trees using a trained ML model 222. The technique and system for performing 3D reconstruction of vessel trees is further described in U.S. application Ser. No. 18/320,156 filed on May 18, 2023 and is incorporated in its entirety by reference herein. As illustrated in FIG. 2C, the trained ML model 222 includes three stages: Stage 1 222a, Stage 2 222b, and Stage 3 222c. Stage 1 222a includes a trained convolutional neural network backbone, Stage 2 222b is referred to herein as a trained vessel centerline stage, and Stage 3 222c is referred to herein as a trained radius reconstruction stage.

FIG. 2D shows a process 280 for performing 3D reconstruction of coronary vessel trees using a trained ML model. The process 280 may be performed by the system 100, and further, the process 280 may be performed according to the schematic diagram of the neural network model of FIG. 2C. For clarity, the process 280 will be described with simultaneous reference to elements of FIGS. 1 and 2.

The process 280 includes providing segmented binary angiography images to the pre-processor 232, at a process 282. The segmented binary angiography images may be provided to the pre-processor 232, or clinical angiograms 202 may be provided to the pre-processor 232 and the pre-processor may perform segmentation of the clinical angiograms 202 to generate the segmented binary angiogram images. The pre-processor 232 then applies a Euclidean distance transform to encode a 2D diameter of each branch of a vessel tree to the binary angiography images, at a process 284. The pre-processor 232 then generates distance transformed binary angiography images.

The pre-processor 232 provides the Euclidean distance transformed angiography images to a trained 3D vessel reconstruction ML model, at a process 286. The 3D vessel reconstruction machine learning model is a machine learning model trained to generate reconstructions of 3D vessels and vessel trees from angiography images. As previously described, the trained ML model 222 includes three stages for performing 3D vessel tree reconstruction. The first stage, Stage 1 222a, includes a trained classical convolutional neural network backbone for image classification, at a process 288. While described as different stages, it should be understood that the neural network backbone used in Stage 1 may be used to perform operations in Stage 2 222b and Stage 3 222c, or at least to perform one MLP of the Stage 2 222b, and/or Stage 3 222c. Alternatively, each MLP of each stage, and each stage itself, may use a different neural network or neural network backbone to perform each stage independently of each other stage and MLP. Additionally, Stage 2 222b and Stage 3 222c may be performed independently and therefore may be performed simultaneously.

The trained vessel centerline reconstruction stage includes a MLP with ReLU activation, at a process 290. The trained vessel centerline reconstruction stage further includes a batch normalization between layers of the MLP for performing centerline reconstruction. In specific examples, the centerline stage, the MLP is composed of 4 hidden layers, where the first 3 layers have 1024 neurons and the last layer has 512 neurons. The trained vessel centerline reconstruction stage takes in the Euclidean distance transformed angiography images and outputs at least a Mᵢ×Nᵢ×3 matrix, with Mᵢ representing the number of branches, and Nᵢ being the number of points in each branch, and 3 being the number of the dimensional coordinates for each centerline point Nᵢ of each branch Mᵢ. The centerline coordinates of the points may be cartesian coordinates, polar coordinates, or another coordinate system to indicate spatial coordinates of the points Nᵢ. Stage 2 222b is perform for each vessel tree and outputs the Mᵢ×Nᵢ×3 matrix for each vessel tree i.

The trained radius reconstruction stage includes a MLP with ReLU activation with batch normalization between layers of the MLP for performing radius reconstruction, at a process 292. The trained radius reconstruction stage is performed for each branch Mᵢ. The trained radius reconstruction stage takes the Euclidean distance transformed angiography images as inputs, and the trained radius reconstruction stage outputs an Nᵢ×1 matrix of radii values, with each radii value corresponding to a respective centerline point Nᵢ. The Nᵢ×1 matrix of radii values are concatenated with the Mᵢ×Nᵢ×3 matrix to form the output Mᵢ ×Nᵢ×4 matrix with spatial coordinates and corresponding radii for each centerline point Nᵢ. In a specific example, the trained radius reconstruction stage includes a separate MLP for each vessel branch, for a total of Mᵢ MLPs for a given vessel tree i. In an example, the radius MLPs are composed of three hidden layers with 128 neurons each, with batch normalization and ReLU activation between each hidden layer. The MLP for each branch may be trained separately to improve the network's ability to capture sudden reductions in vessel radii at regions of stenosis. Without the independent training of each MLP for each branch, stenoses may be overlooked since they make up a small portion of the points in the coronary vessel tree.

The process 280 may further generate and output reconstructed vessel trees 240 from the Mᵢ×Nᵢ×4 matrix, at a process 294. The reconstructed vessel trees 240 may include a low-fidelity vessel tree that includes the branches formed from the centerline points and associated radii at each centerline point. The reconstructed vessel trees may include a high-fidelity vessel tree that includes a volume, with the volume defined by creating a surface spline between outer radii of the radii of each centerline point for each branch of the vessel trees. The low fidelity vessel tree may be referred to herein as a 1D representation of the reconstructed vessel tree, while the high-fidelity vessel tree may be referred to as a 3D representation. To generate the high-fidelity representation, a B-spline, or a non-uniform rational B-spline may be used to form surfaces and define the 3D volume from the Mᵢ×N₁×4 matrix.

FIG. 2E illustrates an example of both a high-fidelity, or 3D vessel tree, representation and a low-fidelity, or 1D vessel tree, representation of a vessel tree. The low-fidelity, or 1D vessel tree model representation, is given by the centerline coordinates and values of radius of each point Nᵢ for each branch Mᵢ. The high-fidelity reconstruction representation includes the volume bounded by the smooth analytical surface formed between the radii at each centerline coordinate point with the volume encompassing all centerline points Nᵢ. In the illustrated example, the vessel tree i is represented by a tree matrix Mᵢ×Nᵢ×4, where Mᵢ is the number of branches in the vessel tree i, Nᵢ is the number of points on each branch centerline, and 4 is the numerical dimension of the data encoded in each point of the branch centerline, specifically its three-dimensional spatial coordinates (x,y,z) and radius r.

FIG. 2F is a schematic diagram of an example implemented architecture of a neural network with angiogram inputs and output 3D reconstructed vessel tree. The multi-stage neural network was designed to reconstruct both vessel centerlines (Stage 2) and radii (Stage 3) for each branch in a coronary tree. The centerline and radius stages both employed a convolutional neural network backbone to train the ML model to prioritize and accurately reconstruct relevant features of the coronary tree from the input images. As an example, a ResNet101 backbone was used as the backbone neural network. While the convolutional layers can learn image-based features relevant to the vessel geometry, a MLP may be better at solving regressions for identifying the 3D coordinates of the vessel centerline points and corresponding radii. Therefore, the final layer of the backbone network included separate MLPs for the centerline stage and radius stage.

In the centerline stage, the final fully connected layer of the backbone network was replaced by a MLP with ReLU activation and batch normalization between layers. The MLP was composed of 4 hidden layers, where the first 3 layers had 1024 neurons and the last layer had 512 neurons. The output of the centerline MLP was a Mᵢ×Nᵢ×3 linear layer, containing Nᵢ centerline points for each of the Mᵢ branches in the binarized angiogram. This output vector was reshaped into a matrix before computing the loss for training.

Meanwhile, the radius stage replaced the final layer of the backbone with a separate MLP for each vessel branch, for a total of Mᵢ MLPs. The radius MLPs were composed of three hidden layers with 128 neurons each, with batch normalization and ReLU activation between each hidden layer. The output of each MLP was a vector of radii of dimension N. The MLP for each branch was trained separately to improve the network's ability to capture sudden reductions in vessel radii at regions of stenosis. Without this step, stenoses may be overlooked since they make up a small portion of the points in the coronary tree.

The radius stage was trained using a mean squared error as a loss function, Eq. 1.

$$\frac{1}{n} {\sum }_{i = 0}^{n} {( {y}_{i} - {\hat{y}}_{i} )}^{2} \qquad Eq . 1$$

In Eq. 1 y is the ground truth, taken as the actual radii of the input vessel tree branches from the input vessel tree, and ŷ is the neural network prediction radii reconstruction. As used herein, “ground truth” values and features refer to the values and features of the input vessel trees, while “predicted” values and features are values associated with the ML model reconstructed vessels, branches, and vessel trees. The centerline stage was trained using the same loss function with an additional vessel length regularization term, Eq. 2.

$$\frac{1}{n} {\sum }_{i = 0}^{n} {( {y}_{i} - {\hat{y}}_{i} )}^{2} + λ \sum {S}_{y} - {S}_{\hat{y}} \qquad Eq . 2$$

In Eq. 2 λ is a regularization rate, S_{y} is an arclength of the ground truth branches, and S_{ŷ} is the arclength of the predicted branches. The vessel length regularization term was included because vessel length is an important determinant of the pressure gradient through a vessel, an important indicator of disease severity.

An Adaptive Moment Estimation (ADAM) optimizer with learning rate 5e-4 and weight decay, L2, regularization was used to train both the centerline reconstruction and the radii stages. The batch size was set to 8 and the multi-stage network was trained for 300 epochs. For the radii stage MLPs, the backbone was frozen after the initial 300 epochs and each branch MLP was trained for an additional 50 epochs.

For comparison, a single-stage neural network model was developed as a counterpart to the multi-stage network described above. The single-stage network architecture was composed of the backbone network and a single MLP which outputs an M×N×4 matrix containing both the centerline points and their associated radii. The single-stage network used a single MLP to determine both the centerline reconstruction and radius reconstruction in a single stage. The loss function of the single-stage network was a weighted mean square error loss function, Eq. 3.

$$\frac{1}{n} {\sum }_{i = 0}^{n} {( {y}_{i} - {\hat{y}}_{i} )}^{2} + {μ ( {r}_{i} - \hat{{r}_{ι}} )}^{2} \qquad Eq . 3$$

In Eq. 3 yᵢ and ŷᵢ represent the ground truth and predicted centerline coordinates while rᵢ and \hat{r}ᵢ represent the ground truth and predicted radii along the centerlines. The regularization parameter u was chosen such that the centerline and radius terms were of the same order of magnitude. The single-stage network was trained using the same hyperparameters as the multi-stage network to make a fair comparison using an ADAM optimizer with learning rate 5e-4, L2 regularization, and a batch size of 8.

To train the proposed multi-staged neural network, hundreds to thousands of ground truth 3D coronary trees can be used with their corresponding segmented 2D angiograms. In practice, this means that thousands of patients with both 3D CTA data and 2D X-ray angiograms must be identified, which is typically not feasible in many medical centers and for many studies. Another challenge of using clinical image data as input for training thew ML model is that the coronaries deform in each frame of an X-ray angiography series due to the contraction of the heart. This necessitates temporal registration of frames from multiple angiographic series in order to create a valid set of input images for 3D coronary tree reconstruction. To produce a large enough dataset and eliminate external sources of error such as temporal registration, a method to produce a sufficiently large training dataset consisting of 5,000 static 3D coronary tree geometries and their corresponding sets of 2D projections was devised. While generated data has been used to train and validate the 3D reconstruction multi-stage neural network described herein, the use of generated projection images as input does not preclude future clinical application. A segmentation algorithm or neural network could be used to convert clinical angiograms obtained during routine patient care in the future into a suitable input for the described 3D reconstruction neural network ML model.

The examples provided focus on 3D reconstruction of the right coronary tree as the large anatomical variation in the left coronary arteries which makes reconstruction more challenging. A method for generating sets of generated angiogram images is implemented. The method includes two steps: 1) a 3D coronary tree generator, and 2) a projection algorithm to create sets of segmented angiograms. The above descriptions of the 3D reconstruction of vessel trees from angiograms is one implementation envisioned, and a person of ordinary skill in the art, would recognize that other implementations including more or fewer stages, and machine learning models, may be implemented to reconstruct 3D vessel trees from angiograms.

In any examples, the described methods, systems, and processes of FIGS. 2C-2F may be used to generate sets of time-series 3D images of vessel trees from clinical angiograms. The set of time-series 3D images may then be provided to the contrast intensity model 206, and the contrast intensity model 206 may determine a number of voxels indicative of the presence of contrast agent in each 3D image. The contrast intensity model 206 may then generate a contrast intensity profile based on the number of voxels in each 3D image indicative of contrast in the 3D image. The contrast image profile may still be a x-y plot with sampling time window as x-axis and number of white pixels in y-axis indicative of the presence of contrast in voxels of the 3D images.

As described above, the microvasculature health model 208 may then obtain the contrast intensity profile and may determine microvascular health parameters (e.g., IMR, CFR, MRR, etc.) from the contrast intensity profile based generated from voxels instead of pixels. Utilizing a 3D image or model and generating contrast intensity profiles from voxels, instead of pixels, preserves three-dimensional flow information that is not obtainable via 2D angiography images. As such, as will be demonstrated further herein, determining CMD via 3D models may be more accurate and allow for higher repeatability of diagnosis than similar methods using 2D images, since the dynamics on contrast agent washout would be independent from the viewing angle of a given 2D image.

### Example Training of Machine Learning Model

FIG. 3A illustrates an example architecture 300 for training a machine learning model that determines microvasculature health of a vessel tree in a vessel inspection region. The architecture 300 includes clinical angiography images 302 and generated angiography images 304, which are derived from a multi-physics model 306, as input data to a segmentation model 308. In various examples, the segmentation model 308 performs image processing including vessel segmentation on the input data to generate segmented images. A contrast intensity model 310 determines contrast intensity profiles from these segmented images, and a microvasculature health model 312 receives these contrast intensity profiles and vasculature health data 314 to train a microvasculature health model 212.

### Training Machine Learning Model Via Generated Data

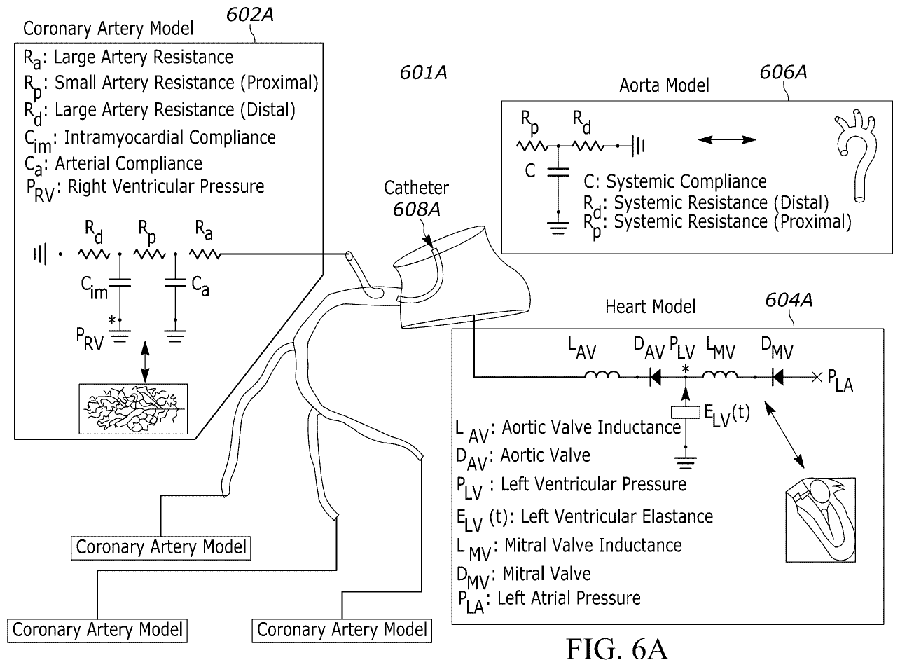

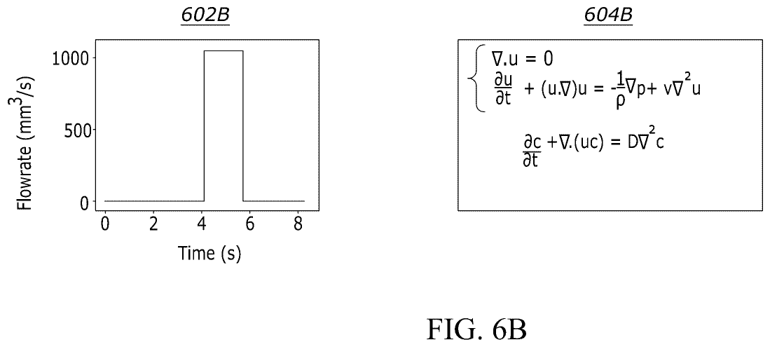

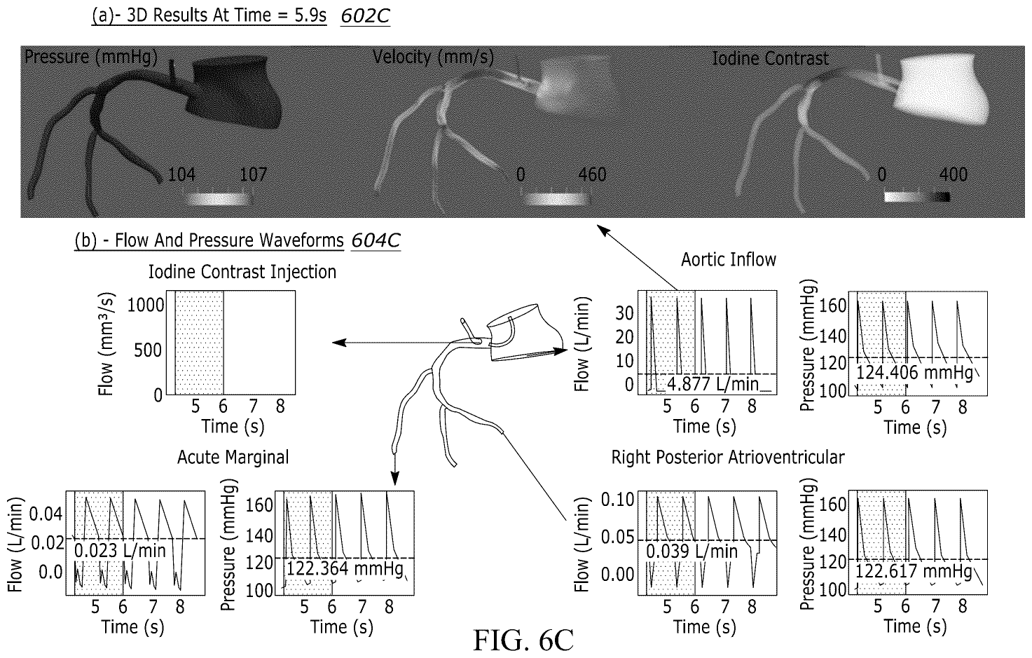

The multi-physics model 306 can be used to simulate clinical angiography processes, i.e., contrast injection and washout in the coronary artery (and corresponding vasculature and microvasculature), to generate a series of generated angiography images 304 to train the microvasculature health model 312. In various examples, the multi-physics model 306 employs lumped parameter models (LPMs) as boundary conditions to produce physiology-relevant pressure and flow patterns. Example LPMs are further described in FIG. 6A. In examples, various flow parameters and metrics, which may include various metrics of the vascular health data 314, can further be determined by the multi-physics model 306. In generating the generated angiography images 304, resistances according to fluid flow models may be provided as inputs to the multi-physics model 306. As such, IMR may be determined and provided to the multi-physics model 306 to generate various series of generated angiography images that correspond to respective IMR values. The multi-physics model 306 may then further generate a series of images simulating contrast injection into vessel with given resistances (e.g., IMR values) to generate sets of 2D images or 3D images of contrast flowing through vessels with known IMRs.

The multi-physics model 306 simulates different instances of contrast injection (e.g., iodine dye) through a vessel tree. The multi-physics model 306 may simulate healthy, moderate disease, and severe disease vasculature and microvascular trees, in addition to various other degrees of vascular health to generate pluralities of generated 3D angiography images 304. Each simulation generates a set of 3D images with each image at a certain time during the simulation of fluid flow through the vasculature. Each plurality of generated 3D images is of fluid flow dynamics of a simulated vessel tree in a vessel inspection region over a sampling time window for a given set of vascular health parameters (e.g., resistances, etc.). For example, if the multi-physics model 306 simulates a healthy vessel tree, then a plurality of generated 3D images is generated for the contrast injection and fluid flow dynamics of the healthy vessel tree over a sampling time window. If the multi-physics model 306 simulates a diseased vessel tree (e.g., buildup of plaques), then a separate plurality of generated 3D images is generated over a sampling time window for the diseased vessel tree. In some embodiments, a single vessel in the vessel tree may be simulated as healthy, diseased, or partially diseased, etc., or groups of vessels or branches may be simulated to generate 3D images of various target vessel regions.

The multi-physics model 306 may use a circuit-based model of blood flow enabling a user to control various flow parameters such as flow resistances of the microvasculature to generate pluralities of the generated 3D images according to various vessel functionalities and degrees of CMD. FIGS. 6A and 7A further provide additional details of an example circuit model of fluid flow in vessels for generating generated 3D images of various degrees of vessel tree health and disease.

The multi-physics model 306 may further generate vasculature health data 314 (e.g., IMR values, MRR etc.) associated with each plurality of generated angiography images of a simulated vessel tree (e.g., health data pertaining to a healthy vessel tree, diseased vessel tree, individual vessel, etc.). The multi-physics model 306 may further include parameters and operations that can compute the IMR value of a vessel tree. For example, parameters such as distal pressure pa and the average transit time of the contrast agent Tₘₙ may be determined from the simulated vessel tree, in which the multi-physics model 306 can subsequently determine a corresponding IMR value. The multi-physics model 306 may then provide the determined IMR values, and any additional flow parameters, to the vascular health data 314, and the generated angiography images 304 and associated vascular health data 314 may then be used to train the microvasculature health model 312. The microvascular health data 314 may include clinically obtained IMR data 314A, generated IMR from one or more simulations 314B (e.g., derived from resistance values of a multi-physics hemodynamic model), hyperemic and baseline IMR values 314A, CFR, MRR, FFR, and other parameters and values indicative of microvascular health. Additionally, the vascular health data 314 may be generated or retrieved from various sources. For example, clinical IMR values 314A may be received from a clinical database, or input by a practitioner from performing an IMR measurement on a patient. The generated IMR values 314B may be provided to the vascular health data 314 by the multi-physics model 306. In examples, the multi-physics model 306 may provide resistance values according to the example circuit models of FIGS. 6A and 7A, or the multi-physics model 306 may calculate one or more parameters indicative of microvascular health (e.g., IMR, hyperemic/baseline IMR 314A, MRR, etc.) from the resistance values and provide the values to be included in the vascular health data 314 for training the microvascular health ML model 312. In examples, any of the vascular health data 314 may additionally be obtained from one or more databases, networks, or memories as required for training the microvascular health model 312.

The image data indicative of dynamics of injection of a contrast agent can be produced through image projection, segmentation, and pixel counting which may be performed in the segmentation model 308, each of which is described further herein. The contrast intensity model 308 then generates contrast intensity profiles for each set of 2D or 3D images. The contrast intensity profiles are then associated with various metrics such as the known IMRs, and used as input-output data pairs to train the microvasculature health model 312, which may include a data-driven machine learning model, to map the relationship between the contrast intensity profiles and the vasculature health data 314.

### Machine Learning Model Training Via 2D Clinical Data

In the illustrated example, clinical angiography images 302 may be obtained and used to generate clinical contrast intensity profiles. As with generated angiography images 304, the broader the patient population of clinical angiography images 302 the larger the training dataset. In practice, generating and obtaining generated data and images may be more readily available and may allow for larger data sets to perform training of a machine learning model. While potentially more limited in number or access, clinical angiography images and clinically obtained health data (e.g., clinically determined IMR, TIMI, CMD metrics, etc.) may be more accurately reflective of actual CMD and the associated flow dynamics in biological systems and patients. As such, it may be beneficial to include clinical angiography images and clinically obtained vascular health data in training the microvascular health model 312.

Vasculature health data 314 (e.g., IMR) for the clinical angiography images 202 may be obtained through performing traditional invasive procedures to determine IMR of patients. The clinical imaging profiles and the vasculature health data 314 can be used as input-output data pairs to train the microvasculature health model 312, which can be a data-driven machine learning model, to map the relationship between the clinical imaging profiles and the vasculature health data 314.

For example, as described previously herein, the segmentation model may perform image segmentation on the clinical angiography images 302 to generate 2D binarized angiography images. The segmentation model 308 may then provide the 2D binarized angiography images to the contrast intensity model 310. The contrast intensity model 310 then performs pixel counting and generates a contrast intensity profile for a given set of binarized angiography images. The contrast intensity profile is indicative of the amount of contrast in a vessel tree over time. The contrast intensity model 310 then provides the contrast intensity profiles to the microvascular health model 312 and vascular health data 314 associated with a set of angiography images is additionally provided to the microvascular health model. The microvascular health model 312 may then be trained using the determined contrast intensity profile for a set of angiography images with the associated vascular health data 314 to train the microvasculature health model 212.

### Examples of 2D and 3D Image Workflows to Perform ML Training

In various examples, the architecture 200 may be used to train the microvasculature health model 312 on 3D images and models, or 2D images whether clinically obtained or generated. FIG. 3B is a schematic diagram of an example system workflow pathway for utilizing 2D images, clinically obtained via angiography and generated via model simulation, to generate contrast intensity profiles for determining microvascular health using machine learning models, and FIG. 3C is a schematic diagram of an example system pathway for utilizing 3D images, generated via multi-physics model and constructed from 2D angiography images, to generate contrast intensity profiles for determining microvascular health using machine learning models.

As illustrated in FIG. 3B, a time-series set of 2D angiography images 302 may be obtained of a patient in a clinical setting. The clinical 2D angiography images are then provided to the segmentation model 208 and segmented 2D images are generated. The contrast intensity model 310 then determines a contrast intensity profile for each set of time-series segmented angiography images based on a number of pixels of each image having contrast agent, as described elsewhere herein.

FIG. 3B also provides a means for generating 2D images from a 3D coronary vessel tree model. For example, the multi-physics model 306 may generate or include a 3D model or an actual heart or arteries of a subject or a vessel inspection region. In particular, in examples herein, the 3D model may be of a vessel tree with a main artery and branches extending therefrom. While the microvasculature is not visible as an explicit part of the generated 3D angiography images, the hemodynamic simulations of the multi-physics model incorporate the functionality and effects of the microvasculature on the flow of blood and contrast agent though the vessel tree. In other examples, the 3D models may include the microvasculature extending from the vasculature.

To generate 3D images (i.e., generated 3D images 304), the multi-physics model 306 may be configured to rotate the 3D model of the vessel inspection region along various axes to provide views of the model from different perspectives. For example, the 3D model may be observed from a top view, side view, back view, etc. each representing different 3D images that can be obtained from rotating the 3D model and following typical viewing angles in the catheterization lab. Such different 3D images may be stored as the generated angiography images 304.

To generate 2D images, the multi-physics model 306 may be configured to manipulate the 3D model and generate 2D images from projecting the 3D model onto different planes, as determined by the multi-physics model 306. In other examples, the multi-physics model 306 may be configured to allow a user to select specific perspective of the 3D model as 2D images. For example, the multi-physics model 306 may take a front view and use that during simulation to generate a plurality of generated angiography images 304. In examples, a dedicated sub-sampler module 305 may receive the 3D generated angiogram images 304 and output 2D generated angiography images of one, or more, angular perspectives of the 3D generated angiogram images 304. For example, the sub-sampler module 305 may perform one or more 2D projections from various perspectives of the 3D generated angiogram images 304 to generate the 2D generated angiograms. The subsampler 305 may then provide the generated 2D angiography images to segmentation model 308. The segmentation model 308 then segments the 2D images and the contrast intensity model 310 further generates one or more contrast intensity profiles from the generated angiography images for further determining microvascular health parameters according to the methods described herein.

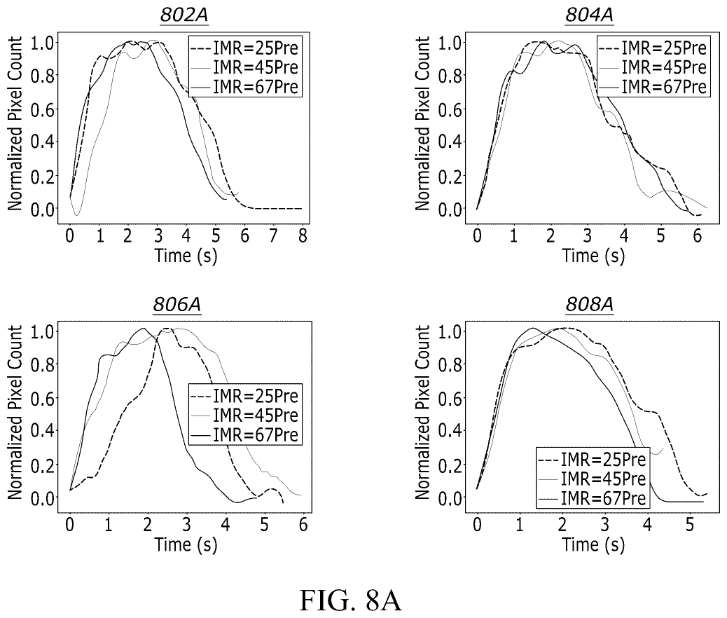

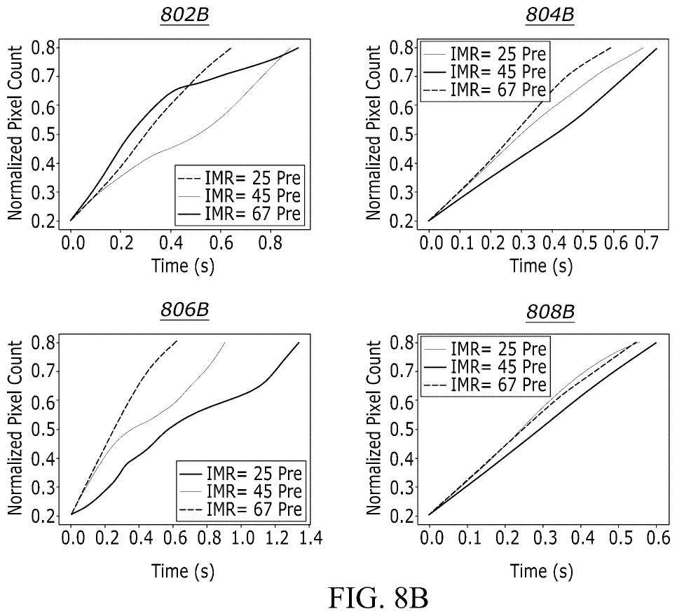

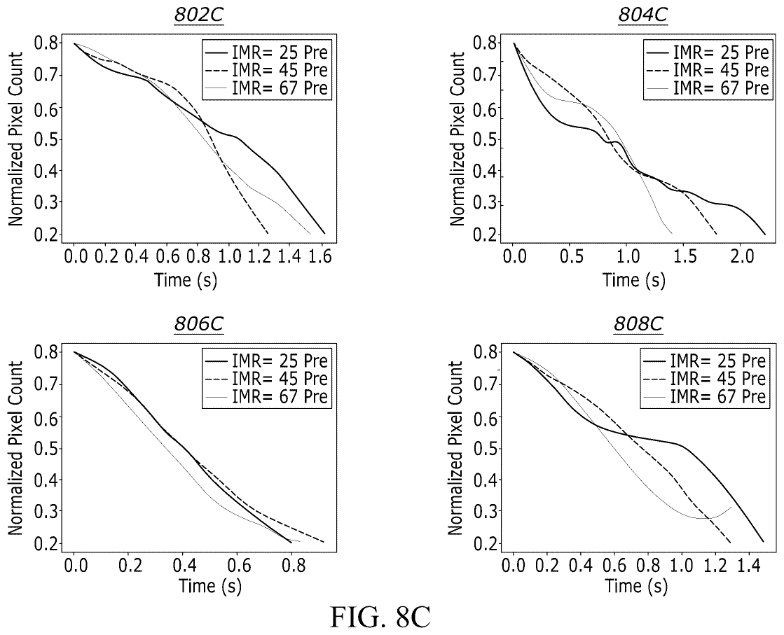

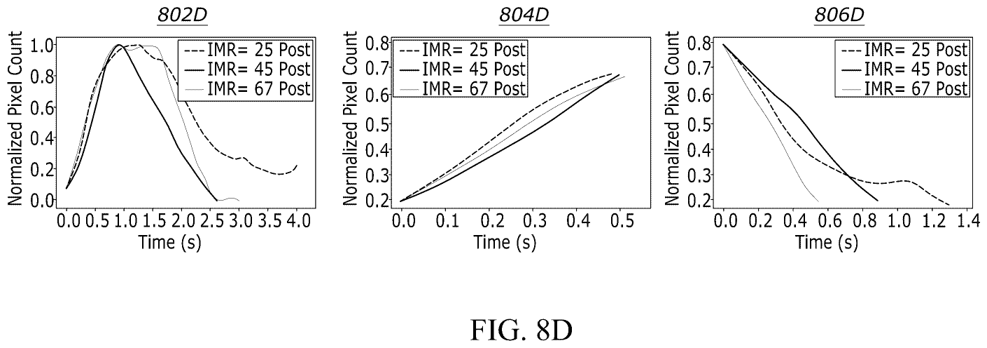

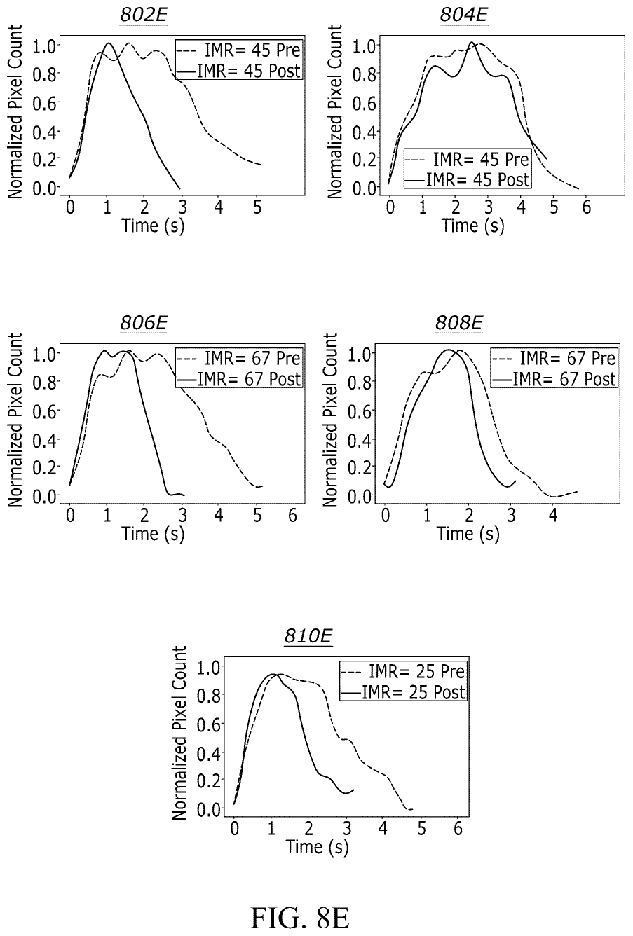

Depending on the perspective (or view) of a 3D image, or resultant 2D image generated from a 3D image, each of the generated angiography images 304, and flow in the images, may vary despite being from the same vessel tree, which can lead to varied contrast intensity profiles. For example, one perspective may generate a contrast intensity profile that indicates a healthy microvasculature but, in another perspective, it may generate a contrast intensity profile that indicates a diseased microvasculature, despite simulating the same vessel tree. Examples of such variance may be shown in FIGS. 8A-8E.

FIG. 3C provides example workflows generating contrast intensity profiles from 3D images and models. The multi-physics model 206 may perform multi-physics simulations, (e.g., hemodynamic flow simulations) and generate multi-physics and provide the 3D generated angiography images, also referred to as 3D images, to the segmentation model 308. The segmentation model then performs image segmentation to further process the 3D images, and the contrast intensity model 310 determines contrast intensity profiles for sets of time-series 3D images. The time-series images show the flow of contrast agent through a coronary vessel tree over a period of time. Further, the contrast intensity model 310 may determine the contrast intensity profiles by determining a number of voxels in each 3D image that includes contrast agent.

In another example, FIG. 3C illustrates generating 3D angiography images of coronary vessel trees from 2D clinical angiography images. A time-series of clinical angiography images is obtained and provided to a 3D image reconstruction model 320 (e.g., the models and methods described with reference to FIGS. 2C-2F). The 3D image reconstruction model 320 then generates a reconstructed 3D image for each angiography image and provides a time-series of reconstructed 3D images to the segmentation model 308. The segmentation model then performing image segmentation and the contrast intensity model 310 determines numbers of voxels indicative of contrast agent in each image and generates a contrast image profile for the time-series of reconstructed 3D images.

The workflows of utilizing 2D and 3D images and models for generating contrast intensity profiles and determining parameters of microvascular health described in reference to FIGS. 3B and 3C are two examples of potential workflows for obtaining and generating 2D and 3D images of coronary vessel trees. Other methods of obtaining, generating, constructing, and reconstructing 2D and 3D images for generating contrast intensity profiles may be implemented for determining parameters of microvascular health using the described methods, systems and ML models.

### Obtaining Clinical Images to Perform ML Training

The clinical angiography images 302 may be obtained from a plurality of vessel inspection regions from different subjects. Each plurality of clinical angiography images 302 can represent clinical angiography images captured over a sampling time window (e.g., full contrast agent injection cycle) through corresponding vessel inspection regions using a contrast injection (e.g., iodine dye). In some embodiments, the certain pluralities of clinical angiography images 302 can be different perspective views of corresponding vessel inspection regions.

Each subject of the different subjects may have different vessel tree conditions. For example, one subject may have a diseased vasculature tree, and a plurality of clinical angiography images can be obtained from a vessel inspection region of the diseased vasculature tree over a sampling time window, while another vessel tree may be healthy and corresponding angiography images of the healthy vessel tree may also be obtained.

The clinical angiography images can be obtained through a medical imaging device such as X-Ray machine. The vasculature health data 314, such as IMR value, for each plurality of clinical angiography images of each patient can be obtained through a traditional, invasive medical procedure such as catheterization.

In some embodiments, some clinical angiography images 302 may be filtered out from training the microvasculature health model. One such scenario may be when different clinical angiography images are obtained from different perspective views of a same vessel tree in a vessel inspection regions. If some perspective views correspond to images indicating a possible diseased vessel tree despite other perspective views corresponding to images indicating a healthy vessel tree, and the vessel tree is actually healthy, then the images that indicate the possible diseased vessel may be filtered out. Likewise, if the vessel tree is actually unhealthy, then images indicating a healthy vessel tree may be filtered out. Other filtering schemes will be appreciated.

Similar filtrations may apply to generated angiography images. Pluralities of generated angiography images can be obtained from different perspective views of a same simulated vessel tree. If one plurality of generated angiography images indicate a better correlation to the actual simulated vessel tree than another plurality of generated angiography images, then the formal generated angiography images may be filtered out.

### Processing and Utilizing the Generated and Clinical Images for Performing ML Training

As discussed, the segmentation model 308 may obtain the clinical angiography images 302 and the generated angiography images 304. The segmentation model 308 then performs segmentation for each image in the clinical angiography images 302 and the generated angiography images 304 to generate segmented images. In examples, the segmentation model 308 may generate segmented images from the clinical angiography images 302 and from the generated angiography images 304 or may use images from both the clinical angiography images 302 and the generated angiography images 304. The segmentation model 308 may include a machine learning model such as a neural network with different convolution layers that has been trained to convert an angiography image into a segmented image. Examples of segmented images are provided in FIG. 4 and FIG. 5.

The contrast intensity model 310 obtains the segmented images from the segmentation model 308 and performs a contrast intensity extraction to generate a contrast intensity profile of the corresponding vessel inspection region over a sampling time window. As such, a contrast intensity profile is generated for each segmented image, and the contrast intensity model 310 determines a plurality of contrast intensity profiles for these segmented images. That is, in some examples, the contrast intensity model 310 assesses segmented images in two steps. First, a contrast intensity extraction determines the total number of illuminated (e.g., white or otherwise exposed) pixels in each segmented image. Second, those total number of illuminated pixels are stored, in a profile manner, across a plurality of segmented images, such as across image frames through washout of the vessel inspection region. The stored profile is a contrast intensity profile. In an example, that contrast intensity profile may be a 2D x-y plot, with the x-axis being the sampling time window in units of time or frame number, and y-axis as the total number or percentage of illuminated pixels in a corresponding segmented image. Details of how to generate a contrast intensity profile are described more in details in the examples of FIGS. 4 and 5.

As described, the vessel inspection region may include a single vessel, or multiple vessels, within a vessel tree. Therefore, it should be appreciated that the techniques described herein in reference to a vessel tree may be performed for a single vessel, such as, for example generating a contrast intensity profile for a single branch rather than for all the vessels in a captured image.

### Determining Microvascular Health via Constructed 3D Images

In some examples, the segmentation model 308 may construct 3D angiography images based on pluralities of 2D angiography images for a same vessel tree. For example, a dedicated and trained machine learning model may take a plurality of angiography images from a same perspective and generate a 3D angiography image of a vessel or vessel tree, as described above. The contrast intensity model 310 may then determine a contrast intensity profile based on the plurality of 3D images with the 3D images forming a time-series of contrast flowing through a target vessel region. In examples utilizing 3D images, the contrast intensity profile may determine a contrast intensity for each image as a number of white voxels out of the total amount of voxels to generate the contrast intensity profile for a given set of 3D images. The contrast intensity model 310 may still provide a x-y plot with sampling time window as the x-axis and number of white voxels as the y-axis.

The microvasculature health model 312 obtains the plurality of contrast intensity profiles from the contrast intensity model 310. In an example, the microvasculature health model 312 may include an encoder 312A and a ML training module 312B. The encoder 312A may reduce each intensity profile in the plurality of contrast intensity profiles to a lower dimension and determine feature maps of the intensity profile. The ML training module 312B may then use the feature maps and the corresponding vascular health data 314 (e.g., IMR, CFR, MRR values) to map the relationships between the feature maps and the vascular health data 314. The ME Training model 212B then outputs one or more parameters indicative of microvascular health and a vessel health sessor 318 may receive the parameters and further determined CFR, FFR, MRR, etc. based on data and outputs from the microvasculature health model 312.

### Example Determination of Contrast Intensity Profile from Clinical Angiograms

FIG. 4 illustrates an example method of determining a contrast intensity profile using clinical angiography images 402. FIG. 4 provides clinical angiography images 402A-402E of a vessel tree in a vessel inspection region over a sampling time window for a certain perspective of the vessel inspection region during which a contrast agent has been injected into the vessel tree. Segmented Images 404A-404E are generated from the clinical angiograms 402. The segmented images are binarized to show the presence of contrast agent (white portions and pixels), as compared to the absence of contrast agent (black portions and pixels) in the vessel tree region. A pixel intensity count is determined as the number of white pixels in each segmented image 404, and the contrast intensity profile 406 is generated as the number of white pixels over time in the time-series of the plurality of angiography images 202. The example contract intensity profile 406 is a x-y plot with a cartesian coordinate system representing white pixel count per segmented image at different times given by frame number. FIG. 4 is an illustration of 5 clinical angiography images and 5 generated, corresponding segmented images, although the number of clinical angiography images and segmented images may include more images (e.g., 50, 100 etc.).

In FIG. 4, the contrast intensity profile 406 is determined from clinical angiography images 402. The clinical angiography images 402 are generated by cardiac catheterization of contrast injection and then obtaining dynamic X-ray pictures of a vessel tree or a single vessel in a vessel inspection region over a sampling time period. Next, the clinical angiography images 402 over the sampling time are supplied to a segmentation model to segment the portion of the vessels occupied by the contrast agent (e.g., iodine contrast) and generate segmented images 404 (e.g., binarized images). The segmentation model can be an angiographic processing network that performs segmentation and performs further processing to fix or address potential errors and imaging issues such as increasing contrast of low-contrast images, considering overlapping catheters, and bony structures, increasing resolution, etc. The segmentation model may aid in generating contrast intensity profiles through recursive segmentation of the time series clinical angiography images. Then, the white pixels in the segmentation images 404 are counted for each frame (404A, 404B, 404C, etc.), and a contrast intensity profile (e.g., contrast intensity profile), representing the injection and washout of the contrast agent is produced from the number of white pixels in each frame. The shape of the resultant intensity profile is indicative of the severity of microvascular disease or IMR. The shape of the intensity profile and correlation to microvascular health is further described in FIGS. 7A-7D.

Each point of the contrast intensity profile 406 is determined from a segmented image 404. Each point represents a number of illuminated (e.g. white) pixels in a given segmented image frame during the sampling time period. For example, the segmented image 404A may be at frame (time) 12 of the contrast intensity profile 406. As shown in the contrast intensity profile 406, the normalized pixel count value is low at about 0.1 as the segmented image 404A does not have a lot of white pixels. The segmented image 404B may be at frame 20 of the contrast intensity profile 406. As shown in the contrast intensity profile 406, the normalized pixel count value is higher with about 0.6 as the segmented image 404B has a lot of white pixels. The segmented image 404C may be at frame 28 of the contrast intensity profile 406. As shown in the contrast intensity profile 406, the normalized pixel count value is very high with about.9 as the segmented image 404B has even more white pixels. The segmented image 404D may be at frame 32 of the contrast intensity profile 406. As shown in the contrast intensity profile 406, the normalized pixel count value at frame 32 is lower than the peak, at about 0.7 as the segmented image does not have as many white pixels as the segmented image 404C. The segmented image 404E may be frame 42 of the contrast intensity profile 406 where the normalized pixel count value is low at about 0.15 as the segmented image 404E has only few white pixels remaining.

### Example Determination of Contrast Intensity Profile from Generated Images

FIG. 5 illustrates a method 500 of determining a contrast intensity profile using 3D generated angiography images 502, through a subsampler process. The method 500 may use generated 3D images 502A-502E that represent the dynamics of a contrast agent (e.g., iodine) in a vessel tree of a vessel inspection area with over a sampling time window. In FIG. 5, the 3D generated angiography images 502 are sub-sampled at certain perspectives (e.g., front view, etc.) to generate 2D sub-sampled images 504A-504E, and the segmented images 506A-506E may be binarized segmentations of the 2D sub-sampled images 504A-504E. A contrast intensity profile 508 may then be generated from the segmented generated angiography images 506 and may be represented as a x-y plot with cartesian coordinates representing white pixel count per segmented image at different times or frames. It should be noted that contrast intensity profiles could be generated directly from the 3D generated angiography images without going through a subsampler process. This approach makes the interpretation of the contrast agent washout independent from the viewing angle.

A segmentation model may obtain the 2D sub-sampled images 504 from a memory or network. The segmentation model may then perform binarized segmentation for each image to generate segmented images 506 of the vessel tree. As such, each 2D sub-sampled image 504A-504E corresponds to a respective segmented image 506A-506E. The segmentation model, in various examples, may begin with obtaining or generating 3D image data, generated via a time series 3D reconstruction of a plurality of recorded 2D images. In some examples, the segmentation model may derive segmented 2D image data from generated 3D angiography data (e.g., via projection of one or more 3D images generated by the multi-physics model). In other examples, the segmentation model can generate segmented 2D image data that are further used to generated contrast image profiles or that is converted to segmented 3D data, for example, a segmented 3D volume.

A contrast intensity model receives the segmented images 506 from the segmentation model and performs a contrast intensity extraction on each of the segmented images 506 to generate the contrast intensity profile 508 of the vessel tree over the sampling time window. The contrast intensity profile may be a x-y plot with cartesian coordinates where the x-axis is the sampling time window and y-axis may be the number of white pixels in a segmented image for a given time.

Each segmented image is a frame indicative of a number of white voxels represented by a point of the plot. For example, the segmented image 506A may be at a time of 4.5 s of the contrast intensity profile 508. As shown in the contrast intensity profile 508, the voxel count value is at about 5000 as the segmented image 506A at time 4.5 s. The segmented image 506B may be at a time of 5 s of the contrast intensity profile 508. With a voxel count of about 12500 as the segmented image 506B has more white voxels than the segmented image 506A. The segmented image 506C may be at a time of 6 s of the contrast intensity profile 508 with a voxel count value of about 19000, corresponding to even more white voxels in the segmented image 506C. The segmented image 506D may be at a time of 6.5 s of the contrast intensity profile 508 with about 15000 white voxels. The segmented image 506E may be at a time of 7 s of the contrast intensity profile 508 with a voxel count value at about 7000 as the segmented image 506E has only few white voxels.

### Computational Multi-Physics Model for Generating Angiography Images and Contrast Intensity Profiles

FIG. 6A-6C are diagrams illustrative of a method of using a multi-physics model to generate 3D angiographic images. FIG. 6A shows a multi-physics model 601A, or CFD model, with a lumped parameter models (LPM) that are used as boundary conditions for simulations. The lumped parameter models may include coronary artery model 602A representing each coronary artery, heart model 604A representing left or right heart, and aorta model 606A representing aortic outflow. FIG. 6B provides a step function 602B for simulation injection and mathematical equations 604B representing Navier-stokes and convection-diffusion equations within 3D coronary artery to calculate velocity, pressure, and concentration of a contrast agent (e.g., iodine contrast). FIG. 6C provides illustrations of simulated hemodynamics and flow transport results 602C at a specified time of pressure, velocity, and iodine contrast of the multi-physics model and flow and pressure waveforms 604C of the multi-physics model. The shaded regions in the plots of 604C indicated timing of injecting contrast into the hemodynamic model.

A novel multi-physics model of iodine contrast injection can provide an interpretation of the parameters that control the different scenarios of the contrast injection and washout. FIG. 6A shows an anatomical model of the aortic root and the main branches of the right coronary artery (RCA) built from a CT scan of a patient. The model includes the distal end of a catheter 608A that can be used to simulate contrast injection.

An aspect of the multi-physics model is the lumped parameter models (LPMs) for the left or right heart 604A, right or left coronary arteries 602A, and aortic outflow 606A which are used as boundary conditions for the multi-physics simulation. To achieve pulsatile aortic flow and pressure, the left heart lumped parameter model 604A can be applied at the aortic inlet. It can consist of five elements, including aortic valve inductance L_{AV}, aortic valve D_{AV}, left ventricular elastance E_{LV}, mitral valve inductance L_{MV}, mitral valve D_{MV}. The heart model may rely on a time-varying elastance function E_{LV} (t), which can represent ventricular contractility. The elastance function can define the relationship between the volume and the pressure of the left ventricle and can produce pulsatile flow and pressure. The period of the elastance function also determines the time of the cardio cycle (e.g., 0.86 seconds). The upper outlet of the aortic root is connected to an aorta model 606A, or a three-element lumped parameter model. The three-element lumped parameter model consists of three elements, which include systemic compliance C, proximal systemic resistance Rₚ, and distal systemic resistance R_{d}. Furthermore, the coronary outlets may be connected to the coronary artery model 602A. The coronary artery model 602A may be more complex than the aorta model 606A, accounting for the systolic myocardial compression that produces the characteristic diastolic-dominated coronary flow patterns. It may include large artery resistance Rₐ, proximal small artery resistance Rₐ, distal small artery resistance R_{d}, intramyocardial compliance Cᵢₘ, and arterial compliance Cₐ. The right ventricle pressure P_{RV} can be applied to the coronary model 602A to produce the diastolic-dominated coronary flow. The right ventricle pressure may be computed by multiplying a factor of ⅓ to the left ventricle pressure P_{LV}.

To simulate the catheterization and the injection of the contrast agent, a catheter 608A can be modeled in the 3D computational domain. The catheter inlet employs a time-varied Dirichlet boundary condition to simulate the injection of contrast agent into the multi-physics model of the coronary vessel tree. Specifically, a step function 602B may be used to simulate the injection of the contrast agent by the cardiologist, as shown in FIG. 6B.

In an example, iodine solution, with a concentration of 400 mol/mm3 and a diffusivity of 0.00203 mm3/s, was used as a contrast agent. In total, two milliliters of iodine solution was injected into the coronary artery within a period of two cardiac cycles (1.72 s). The computation of the transport of contrast within the coronary vessels can provide a surrogate of the clinical angiographic data, and may be used to generate computational data to train the data-driven machine-learning model (microvasculature health model) for inference of IMR, CFR, MRR or other indices of microvascular health.

The models described and demonstrated in FIGS. 6A-6C may employ a finite element method to solve the underlying incompressible Navier-Stokes and convection-diffusion equations within the 3D coronary artery to calculate velocity, pressure, and concentration of iodine contrast as represented in the equations 604B of FIG. 6B. In the provided equations 604B of FIG. 6B, u is the velocity vector field, p is the pressure field, ρ is the fluid density, ν is the kinematic viscosity, c is the concentration of the contrast agent, and D is the diffusion coefficient. Following a mesh-independence analysis, the computational domain of the coronary artery is discretized into approximately 834,853 cells. An internal multi-physics solver (e.g., CRIMSON) may be used to solve the governing equations.

The simulation was performed for 8.6 s, corresponding to ten cardiac cycles and the time step size was set to 0.0001 s. The simulation results were extracted from the sixth cardiac cycle to the end (4.3-8.6 s), leaving the first five cardiac cycles for the numerical solution stabilized.

FIG. 6C depicts flow and pressure waveforms of the multi-physics simulation at aortic inlet, acute marginal and right posterior atrioventricular coronary outlets, as well as the 2-second iodine contrast injection through the catheter as described with reference to FIGS. 6A and 6B. Cardiac output (CO) was 4.88 L/min, and total RCA flow was 0.1 L/min (2% of CO). The coronary outlets present the typical diastolic-dominated profiles. Pressure ranges from 170-100 mmHg, without gradients through the RCA vessels, are consistent with a lack of epicardial disease. The 3D results 602C of FIG. 6C show a snapshot of pressure, velocity, and contrast agent concentration at time t=5.9 s. According to the results of FIG. 6C the hemodynamic results are physiologically relevant and may produce a foundation to study hemodynamics in these patients based on the multi-physics simulations.

### Results and Validation of Trained Machine Learning Models

FIG. 7A illustrates examples of contrast intensity profiles 702B from generated angiography images from different multi-physics simulations of hemodynamics and iodine transport, generated by assigning different values of microvascular resistances to the coronary artery circuit model 704A. The contrast intensity profiles 702A include rising or increasing slopes 712, and decreasing or falling slopes 714, further described in examples in FIG. 7B. The coronary artery circuit model 704A may represent the coronary artery model 602A of FIG. 6A. The three different contrast intensity profiles 702A correspond to varying contrast image profiles 706A, 708A, and 710A of different subjects. The contrast image profile 706A may indicate that a subject has a severe microvascular disease, the contrast image profile 708A may indicate that a subject has a moderate microvascular disease, and the contrast image profile 710A may indicate that a subject has good microvascular health. The various slopes, overall width, maximum width, and other features of the contrast intensity profiles 702A may be used to determine the microvascular health or degree of CMD.

To demonstrate the impact of altered microvascular resistances in the contrast injection curves, the parameters governing coronary microvascular resistance (Rₐ, Rₚ, R_{d} in coronary artery model 704A) were gradually altered to simulate increased degrees of microvascular dysfunction. The contrast intensity profiles 702A correspond to three simulation scenarios: 1) a healthy baseline 710A with a total RCA flow of 0.15 L/min; 2) a moderate disease 708A with a total RCA flow of 0.08 L/min, obtained by uniformly increasing Rₐ, Rₚ, R_{d} by 100% relative to the baseline case; 3) a severe disease 706A with a total RCA flow of 0.05 L/min, obtained by further uniformly increasing Rₐ, Rₚ, R_{d} by 50% relative to the moderate disease case.

A large slope may indicate that the blood flow through a vessel inspection region of a vessel tree may flow fast and without much resistance, which may indicate a good microvasculature health. The contrast image profile with small slope, on the other hand, may indicate that the blood flow through the vessel inspection region may flow slow and due to larger resistance, which may indicate diseased microvasculature health. As shown in contrast intensity profiles 702A, coronary artery model 704A with higher resistance values had smaller slopes. For example, the contrast image profile 706A with high resistance values has smaller slopes compared to the contrast image profile 710A with lower resistance values. These simulated contrast intensity profiles match with the patterns from contrast intensity profiles derived from clinical angiograms, which demonstrate that higher resistance leads to smaller slopes in a contrast intensity profile, as shown in FIG. 7B.

FIG. 7B illustrates contrast intensity profiles 702B and 704B generated from clinical angiography images obtained from actual patients. The contrast intensity profiles 702B represent rising slopes of the contrast intensity profiles. This may indicate a period of time in which the contrast agent is injected into a vessel tree of a vessel inspection region, and when it starts to leave the vessel tree. The contrast intensity profiles 704B represent falling slopes of the contrast intensity profiles. This indicates a period of time in which the contrast agent starts leaving the vessel tree to when it completely leaves the vessel tree.

According to vasculature health data, the contrast intensity profiles with higher slopes correlated with greater IMR values, indicating healthy vessel trees. However, the contrast intensity profiles with smaller slopes correlated with smaller IMR values, indicating unhealthy or diseased vessel tree. Such results may be explained by the fact that microvasculature with higher resistance due to plaque buildups, etc. may slow the flow of blood through the microvasculature, which may lead to more time for the blood to go through the microvasculature.

FIG. 7C illustrates contrast intensity profiles generated for individual coronary arteries: left anterior descending (LAD) and obtuse marginal (OM), depicting contrast uptake and washout for those individual vessels. These vessels may receive a coronary artery bypass 750 to restore adequate blood flow to their respective distal circulations. The contrast intensity profiles for each vessel prior to receiving bypass surgery are indicative of their state of microvascular health and therefore their potential for the bypass to remain open (patent). Long-term patency may be evaluated with a follow-up angiographic assessment 752.

FIG. 7D depicts examples of two CIPs for two different coronary vessels in the same patient: one (upper left side, 754) for the obtuse marginal (OM), the other (upper right side, 756) for the right posterior descending artery (RPDA). The downslope 754A in the OM is steeper than the downslope 756A in the RPDA (blue lines). The upslope in each CIP is indicated in red lines, 754B and 756B, respectively. Over time, coronary artery bypass grafts performed on both vessels resulted in long-term patency of the graft for the OM (therefore indicating a good outflow and state of microvascular health) and failure of the graft for the RPDA (indicating poor outflow and state of microvascular health). The table shows statistically significant differences between the downslope of patent (−5425) vs occluded (−4009) bypasses in different coronary vessels. Therefore, as we show features in the CIP are predictive of the state of microvascular health. In particular, in the illustrated example we show a strong correlation between the downslope in CIP data and microvascular health, where in this example that health corresponds to the efficacy of bypass surgery in the vessel. Vessels characterized by certain downslope characteristics result in better bypass performance over time, in comparison to those vessels characterized by other downslope characteristics.

FIG. 7D illustrates an example application of the present techniques to examine downslope characteristics of the CIP as a biomarker of microvascular health, in particular, as indicated by bypass (patency) efficacy. Thus, in various examples of the present techniques where we describe determining and/or providing CIP to a microvasculature health model, we note that, for at least some biomarker indicators, only a portion of the CIP can be determined and/or provided to the microvasculature health model. In some such examples, CIP data may be determined and only the downslope portion thereof may be provided to the microvasculature health model. In yet other examples, the CIP data may be partitioned into an upslope region, a plateau region, and a downslope region, for example, using data processing techniques. Partitioning the CIP data may allow for providing each data region separately to the microvasculature health model for assessing biomarker status of the respective region. Partitioning the CIP data may allow for data smoothing, filtering, linearization, etc. to be performed on one or more of the respective partitioned regions. Further still, while the CIP data, once determined may be partitioned, in various examples, the CIP data determined itself corresponds to only one of the regions, such as the downslope region.

FIGS. 7E and 7F provide plots and tables presenting results of determined resistance values of a multi-physics model using the trained microvascular health machine learning model.

In FIG. 7E, for example, 600 multi-physics simulations were performed, and health data was generated and used to train the microvasculature health model, or data-driven machine learning models described herein. The dataset was sampled using Latin Hypercube Sampling (LHS) and partitioned as follows: 85% of the data was utilized for training the model, 5% served as validation data for monitoring and fine-tuning the model during training, and the remaining 10% were reserved for testing the model performance post-training. The performance of the data-driven machine learning model was first evaluated against the testing dataset. A plot 702C compares the IMR predictions by the machine learning model and multi-physics simulations. A table 704C shows mean squared errors (MSEs) between the machine learning model and CFD simulations. Results show that the predicted IMRs closely agree with the IMRs computed from CFD simulation for a wide range of IMRs.

In FIG. 7F, for example, 600 multi-physics simulations were performed representative of hyperemic conditions, and an additional 100 multi-physics simulations were performed representative of baseline conditions. Health data was generated and used to train the microvasculature health model, or data-driven machine learning models described herein. The dataset was sampled using Latin Hypercube Sampling (LHS) and partitioned as follows: 85% of the data was utilized for training the model, 5% served as validation data for monitoring and fine-tuning the model during training, and the remaining 10% were reserved for testing the model performance post-training. The performance of the data-driven machine learning model was first evaluated against the testing dataset. A plot 706C compares the CFR predictions by the machine learning model and multi-physics simulations. A table 708C shows mean squared errors (MSEs) between the machine learning model and CFD simulations. Results show that the predicted CFRs closely agree with the CFRs computed from CFD simulation for a wide range of CFRs.

### Examples of Hyperemic and Baseline Contrast Profiles at Different Viewing Perspectives

FIG. 8A represents a pre-hyperemia state of microvascular measurement measured at different perspectives or viewing angles. Pre-hyperemia state may be a baseline condition of the microvasculature before any intervention is made to increase blood flow. Measurements taken in this state may provide a reference for normal blood flow and vascular resistance.

Since a vessel tree in a vessel inspection region of a patient is observable at different perspectives, pluralities of angiography images at different perspectives may be taken and used to output contrast intensity profiles at different perspectives. For example, contrast intensity profiles 802A may be generated by using pluralities of angiography images of three patients at a front view perspective. Contrast intensity profiles 804A may be generated by using pluralities of angiography images of three patients at a side view perspective.

As it can be noted, the contrast intensity profile varies widely depending on the perspective in which the pluralities of angiography images are taken. The lower the IMR value, the lower the resistance within the coronary microcirculation, indicating a good microvasculature health. As one can be expected from FIG. 7B, lower IMR should indicate a higher slope compared to higher IMR. However, a contrast intensity profile 806A, may output result that does not follow such trend. This indicates that there's a variability in the contrast intensity profile depending on the perspective.

One way to reduce such variability may be to combine pluralities of 2D angiography images at different perspectives and reconstruct a plurality of 3D angiography images. Another way to reduce variability may be to selectively choose a perspective that accurately depicts the IMR and filter out other pluralities of angiography images of different perspectives that does not accurately depict the state of the vessel tree. Other methods not described in the specification may be used to reduce variability in contrast intensity profiles.

Similar to FIG. 8A, FIG. 8B and FIG. 8C provide plots of contrast profiles representative of pre-hyperemia state of microvascular measurements measured at different perspectives. In FIG. 8B, rising slopes of the contrast intensity profiles taken at different perspectives are shown in plots 802B-808B. In FIG. 8C, falling slopes of contrast intensity profiles taken at different perspectives are shown in plots 802C-808C.

FIG. 8D provides plots of contrast profiles for a microvascular measurement of a post-hyperemia state. The plots of FIG. 8D show complete contrast intensity profiles 802D, rising slopes 804D, and falling slopes 806D of the microvascular measurement. The post-hyperemia state may indicate a condition of the microvasculature after inducing hyperemia. Hyperemia may indicate increased blood flow through the microvasculature. The post-hyperemia state may be achieved through different measures, such as active exercise, a drug or pill that induces increase in blood flow, etc.

As shown in contrast intensity profiles 802D, the time it took for the contrast agent to enter and exit the microvasculature is faster than any of the pre-hyperemia contrast intensity profiles of FIG. 8A. The rising slopes 804D and falling slopes 806D also represent quicker blood flow of the patients compared to pre-hyperemia state.

FIG. 8E are plots providing direct comparisons of pre-hyperemia state and post-hyperemia state of contrast intensity profiles measured at different perspectives for three different patients. As illustrated in each perspective, the contrast intensity profiles of post-hyperemia states have greater slopes along with faster end times, as seen in 802E, 806E, and 810E. This indicates that when a patient is in a post-hyperemia state, the blood flow through microvasculature is stronger and faster, which expected of a post-hyperemia state.

However, different viewing angles for the first two patients lead to nearly undistinguishable contrast intensity profiles, as seen in 804E (compared to 802E) and 808E (compared to 806E). This is an example of how contrast intensity profiles generated from 2D images lack information pertaining to contrast flow in a third spatial dimension which may cause, as shown in 804E and 802E, for example, errors in distinguishing between pre- and post-hyperemia states, and further cause inaccurate determinations of microvascular health parameters.

### Example Machine Learning Microvascular Health Model

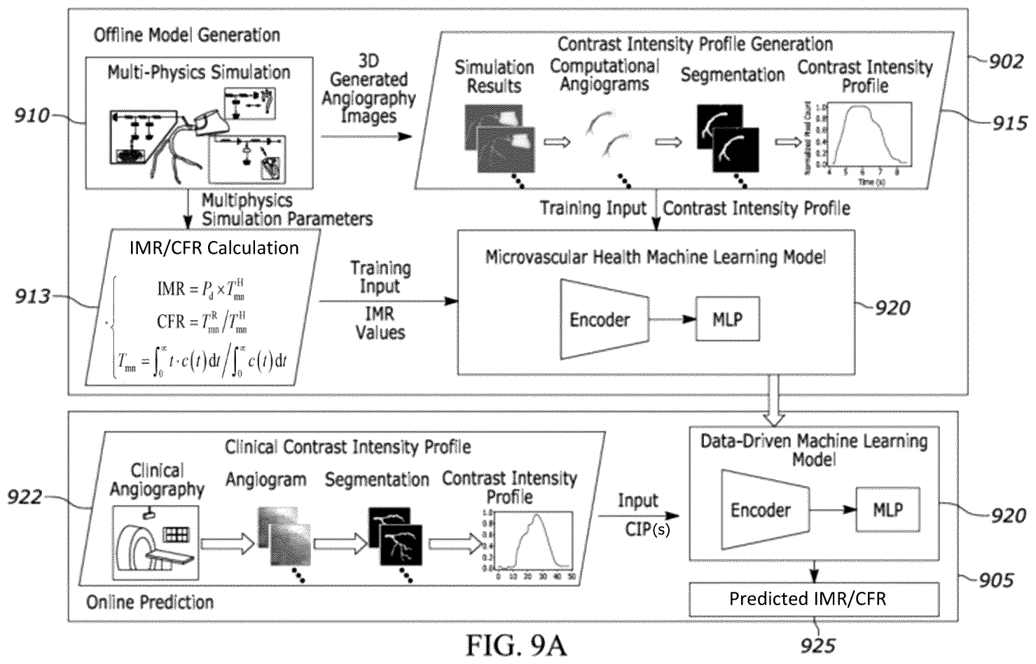

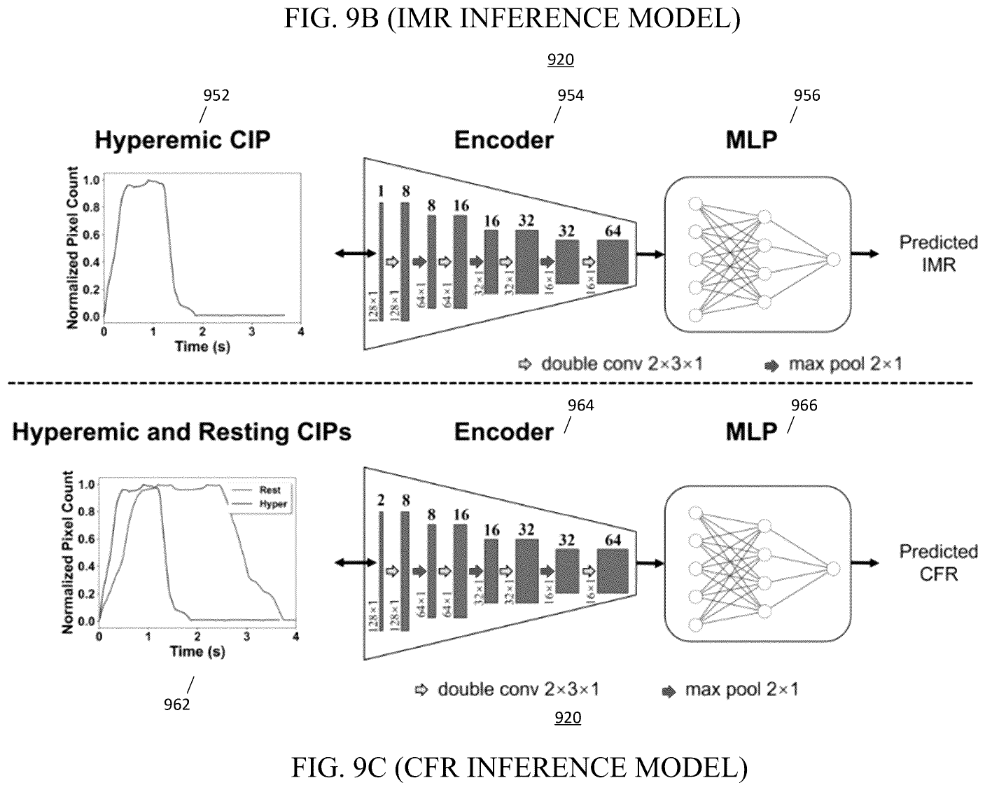

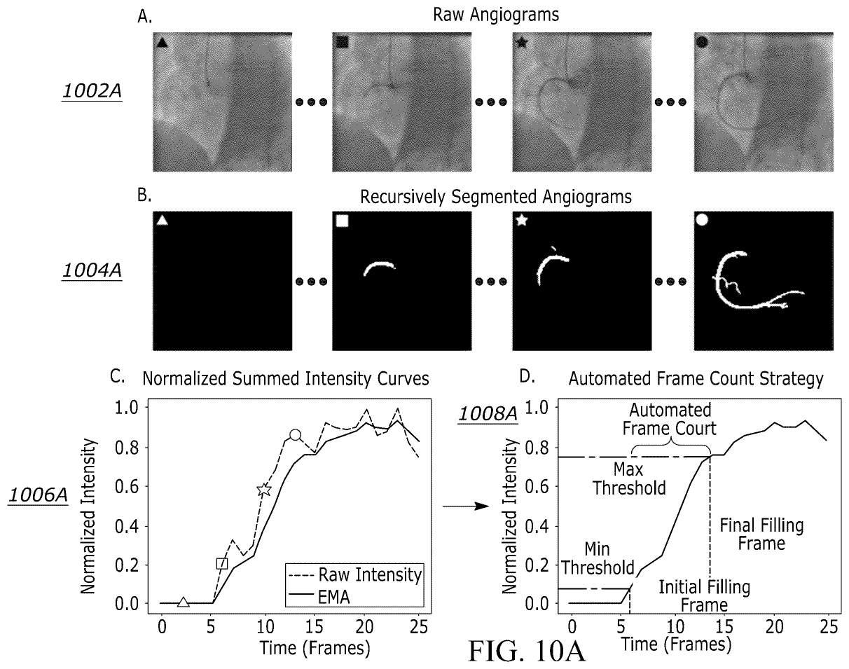

FIG. 9A presents a flow diagram of both machine learning training framework 902 and trained machine learning framework 905 for determining microvascular health parameters from 2D and 3D images as described herein. The machine learning training framework 902 includes performing multi-physics simulations 910, using vessel tree models derived from data of coronary vessel trees, and further uses resistance and capacitance values (as shown in FIGS. 6A and 7A) to simulate flow of blood, and specifically contrast agent flow, through the vessels of the coronary artery model. The multi-physics model provides the input resistance and capacitance metrics to an IMR/CFR calculator stage 913 that uses the various values to determine IMR, CFR, and potentially other parameters indicative of microvascular health (e.g., MRR, etc.). The IMR/CFR calculator stage 913 then provides the determined IMR/CFR values, and any other microvascular health parameters (e.g., MRR, etc.), to a microvascular health ML model 920 as training input.

The multi-physics model 910 further provides generated 3D images (e.g., time-series of 3D generated angiographic image frames) showing contrast flow through the coronary artery model, to the contrast intensity profile generation stage 915. The contrast intensity profile stage 915 receives the generated 3D images from the multi-physics model 910 and generates segmentation images of the 3D images, which may further include downsampling the 3D images into 2D generated angiograms (e.g., by a sub-sampler, via projection, etc.). Contrast intensity profiles are then determined for each image of the segmented 3D images, or segmented 2D generated angiograms. The contrast intensity profile generator stage 915 then provides the generated contrast intensity profiles to the microvascular health ML model 920.

The microvascular health ML model 920 is then trained using the input contrast intensity profiles and IMR values and/or other parameters indicative of microvascular health. The microvascular health ML model 920 may be trained by providing IMR values that correspond to specific contrast intensity profiles, and the machine learning model may then determine characteristics features of the contrast intensity profiles that are used to determine IMR or CFR values. As such, the microvascular health ML model 920 may be trained using supervised, unsupervised, reinforcement learning, or another type of training for machine learning models.

A flowchart of implementation of the trained microvascular health ML model 920 is provided in the trained ML model framework 905. At a clinical stage 922, clinical angiography is performed on one or more patients and a plurality of angiography images are obtained over a period of time of a region with one or more coronary vessel trees. One or more processors then perform segmentation on the plurality of angiograms and determine contrast intensity profiles for each set of time-series angiography images. The clinical stage 922 then provides the contrast intensity profile(s) to the trained microvascular health ML model 920.

The microvascular health ML model 920 receives the contrast intensity profiles as input and further uses an encoder and trained machine learning model to determine a predicted IMR or CFR 925, and potentially other parameters such as MRR, etc., from the contrast intensity profiles. FIGS. 9B and 9C provide a block diagram of a detailed depiction of a microvascular health model 920, in two different examples. FIG. 9B provides an example of the microvascular health model 920 predicting IMR from a single contrast intensity profile input. FIG. 9C provides example of the microvascular health machine model 920 predicting CFR from multiple contrast intensity profile inputs. In the illustrated examples, the microvascular health model 950 includes an encoder 924 and a machine learning model 956 (e.g., a multilayer perceptron (MLP)), and receive or obtain a contrast intensity profile 952 as input.

A data-driven machine-learning model may be designed to investigate the relationship between the contrast intensity profile 952 and IMR or other indices of microvascular health. The machine-learning model takes the contrast intensity profile (e.g., contrast intensity profile) 952 as input and predicts the IMR (FIG. 9B) or other indices of microvascular health value from the contrast intensity profile 952. The encoder 954 may have a series of convolutional and pooling layers that compress the contrast intensity profiles 952 into feature maps. For example, the convolutional layer can be used for feature map extraction, with a kernel size of 2×1 and a stride of 1. The maximum pooling layer can compress the dimensions of the feature maps, with a 2×1 kernel and a stride of 2. The number of channels may be given on the top of each block in the encoder 954, which may be equivalent to the number of kernels applied in each layer. The size of the feature map may be shown beside each block. The encoder may not be limited to the convolution layer and pooling layer given in the example above.

Upon determining the features maps from the encoder 954, the MLP 956 predicts the IMR (FIG. 9B) or other microvascular health indices based on the feature maps. For example, the MLP can have four layers, with 1024, 1024, 128, and 1 neurons which can be used to output predicted IMR. The MLP may not be limited to just four layers given in the example above.

A data-driven machine-learning model may be designed to investigate the relationship between contrast intensity profiles at baseline and hyperemic states and the CFR, or other indices of microvascular health. The machine-learning model takes two contrast intensity profiles 962 as input and predicts the CFR or other indices of microvascular health value. An encoder 964 may have a series of convolutional and pooling layers that compress the contrast intensity profiles 962 into feature maps. For example, the convolutional layer can be used for feature map extraction, with a kernel size of 2×1 and a stride of 1. The maximum pooling layer can compress the dimensions of the feature maps, with a 2×1 kernel and a stride of 2. The number of channels may be given on the top of each block in the encoder 964, which may be equivalent to the number of kernels applied in each layer. The size of the feature map may be shown beside each block. The encoder may not be limited to the convolution layer and pooling layer given in the example above.

Upon determining the features maps from the encoder 964, an MLP 966 predicts the CFR or other microvascular health indices based on the feature maps. For example, the MLP can have four layers, with 1024, 1024, 128, and 1 neurons which can be used to output predicted CFR and other microvascular health indices. The MLP may not be limited to just four layers given in the example above.

While IMR and CFR are used as an example in FIGS. 9A and 9B, the system and methods described may be used to predict other vascular health parameters such as MRR, FFR, and other parameters in both hyperemic and baseline states.

### Determination of TIMI Via Machine Learning Model and Contrast Profiles

While various examples are described herein for determining microvasculature health, e.g., by assessing IMR and microvasculature disease from contrasty intensity profile data, other measures of microvasculature health may be determined using the present techniques. These include, for example, determining TIMI and from TIMI microvasculature health.

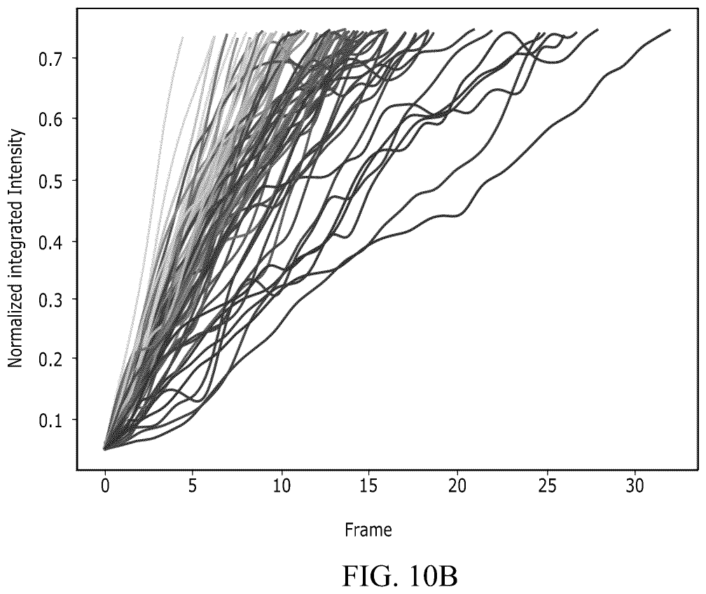

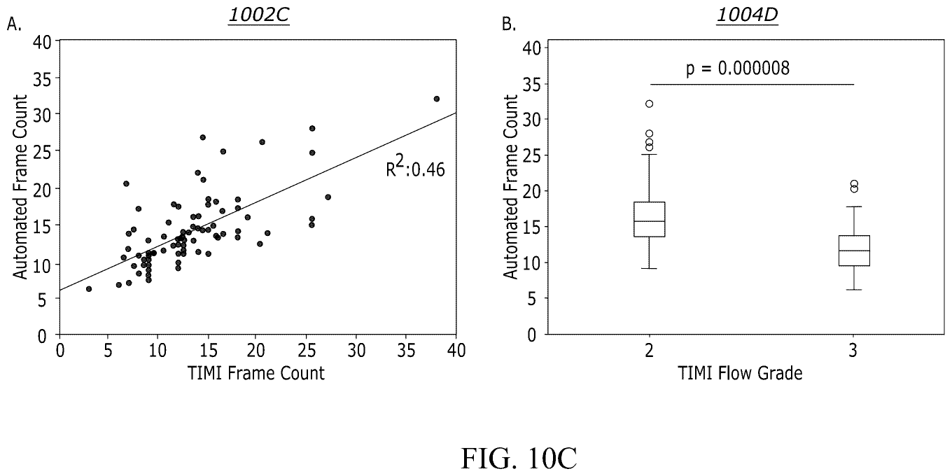

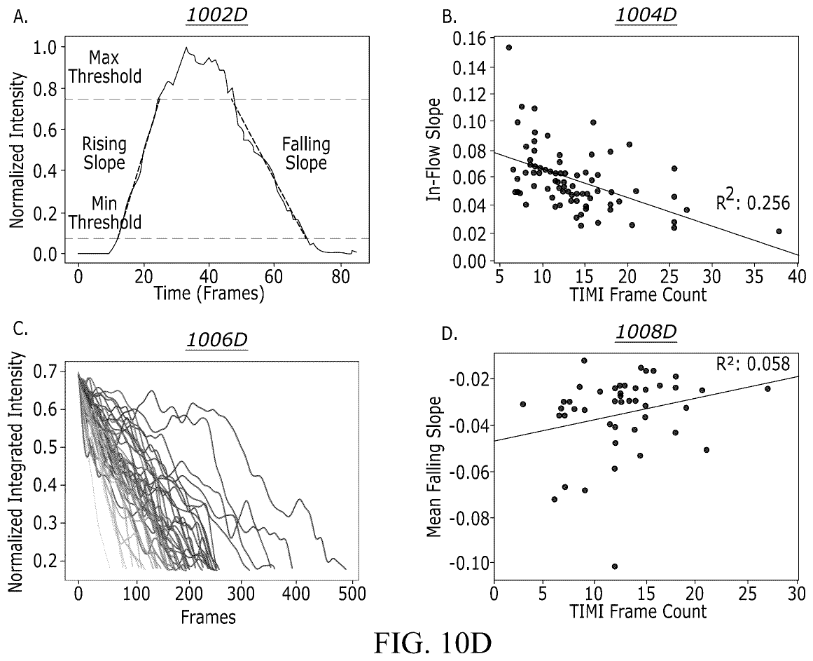

FIGS. 10A-10D illustrate processes of using a segmentation model and contrast intensity model to determine segmentation-based frame counts (i.e., contrast intensity extraction) and contrast profiles, which can demonstrate strong correlations with TIMI frame counts and also provide novel information about coronary microcirculation.

Adequate blood flow through the coronary tree may be critical for maintaining cardiac perfusion. Coronary angiography has the potential to provide rich, dynamic information about coronary hemodynamics. However, current strategies to assess flow through coronaries depend on either subjective expert opinion (TIMI flow grade) or laborious frame-by-frame anatomical analysis (TIMI frame count).

A technique for automated characterization of bulk flow through the coronary tree using the right coronary artery is described for example. The segmentation model (e.g., a segmentation neural network such as described in U.S. application Ser. No. 18/225,363 filed on Jul. 24, 2023 and incorporated in its entirety by reference herein) generates sequential segmentations of angiograms, creating a time series of summed segmentation intensity profiles, and characterizes the filling and wash-out phases of the contrast intensity profiles. The segmentation-derived frame counts and normalized mean filling slopes of inflow dynamics may correlate with manual frame counts and flow grades. The generated outflow dynamics may provide additional information to the traditional frame count and flow grade metrics, which suggests that the segmentation-derived frame counts may capture novel information about the coronary microcirculation.

Coronary angiography may enable the visualization of the coronaries and can provide a foundation for the assessment of anatomic stenosis during heart catheterization. Accurate and objective assessment of flow can provide information about the physiological relevance of stenoses, the adequacy of revascularization, and microvascular function that is likely of prognostic utility.

In clinical practice, flow is frequently described subjectively using the TIMI flow grading which involves pseudo-quantification of flow based on expert evaluation of the bulk filling of the coronary tree. The TIMI Frame Count method was devised to provide a quantitative assessment of flow by counting the number of frames between dye entering the coronary ostium and arriving at pre-determined landmarks. This approach enables assessment of bulk flow but requires labor-intensive manual identification of the relevant time points. Other approaches to quantify flow through the coronary tree have been developed but typically require deployment of pressure sensitive catheters (FFR) or use of additional imaging modalities that provide largely estimates (CT-FFR).

An automated approach to quantitatively assess bulk flow from angiograms facilitated by the segmentation model using the right coronary artery (RCA) may provide quantification of flow that correlates well with TIMI Frame Counts without requiring manual identification of anatomical landmarks in angiograms like those used in the initial training of the network.

The angiograms used in the example provided were a subset of 80 RCA training dataset angiograms included in initial segmentation model training. The full dataset included 462 angiograms (280 images of the left coronary artery and 182 images of the RCA) acquired using a Siemens Artis Q Angiography system. Patients with pacemakers, implantable defibrillators, or prior bypass grafts were excluded, due to concern that exogenous objects would produce artifacts in the segmentation. Patients with chronic total occlusions were also excluded. In the cohort of 161 patients, 14 had severe stenosis (≤80% diameter reduction) and the remaining had mild to moderate stenosis. The subset of RCA angiograms was randomly selected from the 182 RCA angiograms in the full dataset. With low risk for subject harm and retrospective use of data collected for clinical purposes.

Manual review of angiograms involved characterization of the TIMI grade and corrected TIMI frame count, hereafter referred to as the TIMI frame count. TIMI frame counts may be corrected for variations in acquisition frame rate and normalized to 15 FPS. Angiograms were reviewed independently by 2 reviewers in batches of 20 angiograms, with intervening mediation sessions to support consensus on TIMI frame count and grade between batches, until after 2 rounds the Kendall Tau for inter-rater reliability for the subsequent batch was >0.75. The raters then divided up and independently reviewed the remaining angiograms. The Kendall Tau for interrater reliability for the entire cohort was 0.73, corresponding to a correlation coefficient of 0.87.

De-identified angiograms were separated into individual PNG frames (frames 1002A of FIG. 10A). These PNGs can be recursively segmented by the segmentation model (frames 1004A of FIG. 10B). The segmentation model included an Angiographic Processing Network (APN) coupled to a semantic segmentation network Deeplabv3+. The APN was trained to mimic several unsharp mask filters applied in series and the Deeplabv3+ network was initialized with pre-trained weights available from the Tensorflow Deeplab GitHub repository. The coupled network was subsequently trained on a combination of single angiogram frames that were labeled by expert reviewers. Of note, the frames used for training were selected by identifying those with the maximum summed pixel intensity (e.g., those with the most filled coronary tree). While the training dataset included both left and right coronary trees with a diversity of anatomies, it included few critical stenoses.

Time series plots 1006A and 1008A of the net intensity of angiograms were generated by summing the number of filled pixels in each frame 1004A. The intensity time series were smoothed using an exponential moving window with a=0.4 and then normalized to the range [0,1] by subtracting the minimal intensity and dividing it by the maximal intensity. B-spline basis functions were fit to individual nomograms to enable extrapolation of filling time points between individual frames at a resolution of 0.1 frames.

Based on the normalized intensity time series, ‘automated TIMI frame counts’ were calculated as presented in plot 1008A of FIG. 10A. The initiation of filling was defined as the time when the spline curve crosses the ‘minimal filling threshold’, defined as 0.05 which was selected to avoid inclusion of segmentation artifacts. The end of proximal filling was defined as the ‘maximal filling threshold’ and two different strategies for setting the ‘maximal filling threshold’ were explored. First, the maximum filling threshold was set at the mean normalized intensity of the final TIMI Frame Count frame for a subset of 10 angiograms; and second, the maximum filling threshold was tuned to optimize the correlation with the TIMI Frame Count. The automated TIMI frame count was defined as the number of frames between the minimal and maximal filling thresholds and was normalized for acquisition frame rate.

Using the same minimum and maximum filling thresholds, the in-flow slope was defined as the average rise in pixel intensity per frame between the two thresholds, shown in plot 1002D of FIG. 10D. Similar maximum and minimum thresholds were applied to the falling slope of the intensity profile to enable calculation of mean falling slopes. Angiograms in which the minimum filling threshold was not reached during outflow were omitted from the out-flow slope analysis. The generated slopes have units of normalized intensity per frame as shown in the two-dimensional cartesian coordinate axes.

Overall, the segmentation quality for individual angiogram frames was highly effective and successful. However, the plot 1006A of FIG. 10A shows substantial frame-to-frame variability in normalized intensity. The plot of FIG. 10B provides all the rising intensity profiles together and illustrates the large variance in average slope between angiograms. There were also notable non-linearities within individual curves that were observed in the rising intensity profiles.

The correlation between TIMI and segmentation model derived frame counts was analyzed. A line plot 1002C in FIG. 10C shows TIMI frame counts (13.4±5.7 frames, [3-38frames]) plotted on the abscissa against Segmentation-based frame counts (14.2±5.0 frames, [6.2-32.1 frames) plotted on the ordinate. The trend line shows the least-squares regression fit with a correlation coefficient of 0.46 and t-test p-value of 6*10⁻¹². The Kendall Tau statistic for the comparison between automated and manual frame counts was 0.50.

A box plot 1004D of FIG. 10C shows Segmentation-derived frame counts for the each TIMI flow grade included in the angiogram cohort. The distributions of automated frame count for the angiograms with TIMI flow grade 2 (17.0±5.6 frames, [9.2-32.1frames]) and those with TIMI flow grade 3 (12.1±3.3 frames, [6.2-21.0 frames]) can be analyzed with a two-tailed T-test, yielding a p-value of 3*10⁻⁶.

Alternative parameters for characterizing bulk flow from the normalized intensity plots were determined by calculating the mean rising and falling slopes for each angiogram.

The mean slope of the rising intensity profile between the first crossings of the minimum and maximum thresholds was determined as shown in 1002D of FIG. 10D. A line plot 1004D of FIG. 10D shows TIMI frame counts plotted on the abscissa against intensity plot mean in-flow slope on the ordinate (0.059±0.023, [0.021-0.15]). The trend line in the line plot 1004D shows the least-squares regression fit with a correlation coefficient of 0.266 and corresponding t-test p-value of 4*10⁻⁵.

Falling portions of the intensity profiles were also analyzed. The falling intensity profiles 1006D of FIG. 10D show the intensity profiles plotted from the last crossing of the maximum threshold to the last crossing of the minimum threshold. As with the rising portions of the curves, there can be a large qualitative variance in the rate of dye washout and notable non-linearities within individual curves. A line plot 1008D of FIG. 10D shows TIMI frame counts plotted on the abscissa against mean out-flow slope (0.059±0.023, [0.021-0.15]) on the ordinate. The trend line in FIG. 10D shows the least-squares regression fit with a correlation coefficient of 0.045 and corresponding p-value of 0.22.

The provided results of FIGS. 10A-10D demonstrate the feasibility of leveraging automated angiogram segmentation to perform quantitative assessment of bulk flow through coronary trees. This strategy involves segmentation of individual angiogram frames followed by automated quantification of the contrast in-flow frame count and mean slopes of both the filling and wash-out phases. The Segmented-derived frame counts and mean filling slopes of inflow dynamics show significant and, in the case of frame counts, relatively strong correlations with the TIMI frame counts.

The present techniques compare with the currently employed methods for flow quantification from angiograms. In particular, the present techniques can provide a more standardized way to assess flow while calculating novel information on the differences in outflow dynamics.

As demonstrated by various examples herein, the present techniques may be used to identify one or more biomarkers of microvascular health, for example, through predicting vessel responsiveness to various treatments.

For example, the present techniques include methods of assessing microvasculature function of a vessel inspection region and in response predicting a treatment response of that vessel inspection region. Angiography images of a vessel tree in the vessel inspection region may be captured over a sampling time window during which a contrast agent has been injected into the vessel tree. These angiography images may be applied to a segmentation model configured to generate segmented images of the vessel tree and those segmented images may be provided to a contrast intensity model, as described herein. The contrast intensity model may generate a contrast intensity profile of the vessel tree over the sampling time window and provide at least a portion of that contrast intensity profile to a microvasculature health model that predicts a response of the vessel inspection region to a treatment based on a characteristic of the at least a portion of the contrast intensity profile. As demonstrated by various examples herein that portion of the contrast intensity profile may be a downslope region of a profile for some biomarker determinations, although other regions of a profile may be analyzed for other biomarker determinations. The present techniques include generating and storing and/or displaying a report that serves as an electronic indication of the predicted response to the treatment by the vessel tree or by some portion thereof.

Indeed, in various examples herein, where an input data is described as a contrast intensity profile, the input data may be a portion of a determined contrasty intensity profile, such as the downslope portion, any portion isolated from the upslope portion, or any other portion of the profile, that corresponds to training of microvasculature health model.

More specifically, in some examples, obtaining contrast intensity profile data or portions thereof, for subjects prior to treatment and again after a treatment (treatment regimen, combination of treatments, etc.), provides a training data set to train a microvasculature health model. Example treatments include medical procedures such as a bypass procedure, coronary microvascular intervention, mechanical or ultrasound-based thrombectomy, angiogenesis therapy, stenting, and venous occluder. Other treatments include pharmacological treatments such as an antiplatelet agent, an anticoagulant agent, a vasodilator agent, anti-inflammatory agent, a statin, nitroglycerin, calcium channel blockers, beta-blockers, ACE inhibitors, or other anti-anginal medications.

### Additional Aspects

Aspect 1. A method of determining microvasculature function of a vessel inspection region, the method comprising: obtaining angiography images of a vessel tree in the vessel inspection region, the angiography images captured over a sampling time window during which a contrast agent has been injected into the vessel tree, and applying the angiography images to a segmentation model configured to generate segmented images of the vessel tree; providing the segmented images to a contrast intensity model and, performing, by the contrast intensity model, a contrast intensity extraction on each of the segmented images to generate a contrast intensity profile of the vessel tree over the sampling time window; providing the contrast intensity profile to a microvasculature health model configured to determine a health of the microvasculature within the vessel tree based on the contrast intensity profile; and determining, using the microvasculature health model, a microvasculature health of microvasculature of the vessel tree.
Aspect 2. The method of Aspect 1, wherein the segmentation model is a trained machine learning model.
Aspect 3. The method of Aspect 2, wherein the trained machine learning model is a neural network.
Aspect 4. The method of Aspect 2, wherein the segmentation model is configured to generate the segmented images as two-dimensional (2D) segmented images.
Aspect 5. The method of Aspect 2, wherein the segmentation model is configured to generate segmented three-dimensional (3D) images of the vessel tree from the angiography images.
Aspect 6. The method of Aspect 1, wherein the contrast intensity model is configured to determine a pixel metric for each segmented image and to determine a rising trend and a falling trend of the pixel metric over the sampling time window for the segmented images, the contrast intensity profile comprising the rising trend and the falling trend.
Aspect 7. The method of Aspect 6, wherein the contrast intensity model is a machine learning model.
Aspect 8. The method of Aspect 1, wherein the angiography images comprise images of the vessel tree in both (i) a baseline state and (ii) a hyperemic state.
Aspect 9. The method of Aspect 8, further comprising determining, by the contrast intensity model, contrast intensity profiles for (i) angiography images of the vessel tree in the baseline state, and (ii) angiography images of the vessel tree in the hyperemic state, and wherein determining the microvasculature health comprises determining at least one of microvasculature resistance reserve or coronary flow reserve from the contrast intensity profiles of the baseline and hyperemic states.
Aspect 10. The method of Aspect 1, the method further comprising: obtaining additional angiography images of the vessel tree captured over a second sampling time window during which the contrast agent has been injected into the vessel tree, the additional angiography images captured at a different angle than the angiography images, providing the additional angiography images to the segmentation model to generate a second set of segmented images of the vessel tree; providing the second set of segmented images to the contrast intensity model and, performing, by the contrast intensity model, the contrast intensity extraction on each of the second set of segmented images and generating a second contrast intensity profile of the vessel tree over the second sampling time window; and providing the second contrast intensity profile to the microvasculature health model.
Aspect 11. The method of Aspect 1, the method further comprising: obtaining additional angiography images of the vessel tree captured over a second sampling time window during which the contrast agent has been injected into the vessel tree, the additional angiography images captured at a different angle than the angiography images, providing the additional angiography images to the segmentation model to generate a second set of segmented images of the vessel tree; providing the second set of segmented images to the contrast intensity model and, performing, by the contrast intensity model, the contrast intensity extraction on each of the second set of segmented images and generating a second contrast intensity profile of the vessel tree over the second sampling time window; comparing the contrast intensity profile and the second contrast intensity profile to reference contrast intensity profiles to identify one of the contrast intensity profile and the second contrast intensity profile as having a greater correlation to the reference contrast intensity profiles; and providing the one of the identified contrast intensity profile or second contrast intensity profile having the greater correlation to the microvasculature health model.
Aspect 12. The method of Aspect 1, wherein the sampling time window extends from an initial injection of the contrast agent into the vessel tree through washout of the contrast agent from the vessel tree.
Aspect 13. The method of Aspect 1, wherein the microvasculature health model comprises a trained machine learning algorithm trained on training angiography images and at least one of (i) index of micro-circulatory resistance data, (ii) coronary flow reserve data, and (iii) microvascular resistance reserve data corresponding to the training angiography images, multi-physics simulation data corresponding to the training angiography images, and contrast intensity data.
Aspect 14. The method of Aspect 13, wherein the microvasculature health model is trained to generate at least one of an index of micro-circulatory resistance, a coronary flow reserve value, and a microvascular resistance reserve value for the vessel tree.
Aspect 15. The method of Aspect 13, wherein the microvasculature health model comprises an encoder stage for receiving the contrast intensity profile and a multilayer perceptron stage fed by the encoder and trained to generate at least one of a predicted index of microcirculatory resistance, a predicted coronary flow reserve value, and a predicted microvascular resistance reserve value as an indicator of the microvasculature health of the vessel tree.
Aspect 16. A computer-implemented method for training a microvasculature health determination system, the method comprising: obtaining angiography images of a plurality of vessel inspection regions from different subjects, the angiography images including subsets of angiography images captured over a full contrast agent injection cycle through corresponding vessel inspection regions, the angiography images include subsets of angiography images captured at different perspective views of corresponding vessel inspection regions; obtaining vasculature health data for each of the angiography images; performing a segmentation on each of the angiography images to generate a segmented image for each angiography image; providing the segmented images to a contrast intensity model configured perform a contrast intensity extraction on each of the segmented images to generate a contrast intensity profile of a vessel tree in the vessel inspection region over a sampling time window; and providing the contrast intensity profile and the vasculature health data to a machine learning model to train the machine learning model to generate a microvasculature health of a vessel tree in a subsequently imaged vessel inspection region.
Aspect 17. The method of Aspect 16, wherein the vasculature health data comprises at least one of index of micro-circulatory resistance data, fractional flow reserve data, coronary flow reserve data, and microvascular resistance reserve data.
Aspect 18. The method of Aspect 17, the method further comprising: generating angiography images using a multi-physics model of a contrast injection through a vessel tree, the vessel tree having at least one coronary vessel branching into microvasculature vessels, the generated angiography images being binarized, and the generated angiography images corresponding to scenarios (i) baseline health microcirculation with baseline values of microvascular resistance, (ii) moderate disease microcirculation with moderate values of microvascular resistance, and (iii) severe disease microcirculation with high levels of microvascular resistance; providing the generated angiography images to the contrast intensity model for generating contrast intensity profiles; and providing the generated contrast intensity profile to the machine learning model to train the machine learning model to generate the microvasculature health of the vessel tree in the subsequently imaged vessel inspection region.
Aspect 19. A method of assessing microvasculature function of a vessel inspection region for predicting a treatment response, the method comprising: obtaining angiography images of a vessel tree in the vessel inspection region, the angiography images captured over a sampling time window during which a contrast agent has been injected into the vessel tree, and applying the angiography images to a segmentation model configured to generate segmented images of the vessel tree; providing the segmented images to a contrast intensity model and, performing, by the contrast intensity model, a contrast intensity extraction on each of the segmented images to generate a contrast intensity profile of the vessel tree over the sampling time window; providing at least a portion of the contrast intensity profile to a microvasculature health model configured to predict a response of the vessel inspection region to a treatment based on a characteristic of the at least a portion of the contrast intensity profile; and generate an electronic indication of the predicted response to the treatment.
Aspect 20. The method of Aspect 19, wherein the at least a portion of the contrast intensity profile is a downslope of the contrast intensity profile.

Aspect 21. The method of Aspect 19, wherein the at least a portion of the contrast intensity profile is a portion of the contrast intensity profile isolated from an upslope portion of the contrast intensity profile.

Aspect 22. The method of Aspect 19, wherein the microvasculature health model is configured to predict the response of the vessel inspection region to the treatment based on a downslope of the contrast intensity profile.
Aspect 23. The method of Aspect 19, wherein the treatment is a medical procedure selected from the group consisting of a bypass procedure, coronary microvascular intervention, mechanical or ultrasound-based thrombectomy, angiogenesis therapy, stenting, or venous occluder.
Aspect 24. The method of Aspect 19, wherein the treatment is a pharmacological treatment selected from the group consisting of an antiplatelet agent, an anticoagulant agent, a vasodilator agent, anti-inflammatory agent, a statin, nitroglycerin, calcium channel blockers, beta-blockers, ACE inhibitors, or other anti-anginal medications. Additional Considerations

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the target matter herein.

Additionally, certain embodiments are described herein as including logic or a number of routines, subroutines, applications, or instructions. These may constitute either software (e.g., code embodied on a non-transitory, machine-readable medium) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion such as a Contrast Agent Injection System) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

Those skilled in the art will recognize that a wide variety of modifications, alterations, and combinations can be made with respect to the above described embodiments without departing from the scope of the invention, and that such modifications, alterations, and combinations are to be viewed as being within the ambit of the inventive concept.

While the present invention has been described with reference to specific examples, which are intended to be illustrative only and not to be limiting of the invention, it will be apparent to those of ordinary skill in the art that changes, additions and/or deletions may be made to the disclosed embodiments without departing from the spirit and scope of the invention.

The foregoing description is given for clearness of understanding; and no unnecessary limitations should be understood therefrom, as modifications within the scope of the invention may be apparent to those having ordinary skill in the art.

What is claimed:

1. A method of determining microvasculature function of a vessel inspection region, the method comprising:
obtaining angiography images of a vessel tree in the vessel inspection region, the angiography images captured over a sampling time window during which a contrast agent has been injected into the vessel tree, and applying the angiography images to a segmentation model configured to generate segmented images of the vessel tree;
providing the segmented images to a contrast intensity model and, performing, by the contrast intensity model, a contrast intensity extraction on each of the segmented images to generate a contrast intensity profile of the vessel tree over the sampling time window;
providing the contrast intensity profile to a microvasculature health model configured to determine a health of the microvasculature within the vessel tree based on the contrast intensity profile; and
determining, using the microvasculature health model, a microvasculature health of microvasculature of the vessel tree.

2. The method of claim 1, wherein the segmentation model is a trained machine learning model.

3. The method of claim 2, wherein the trained machine learning model is a neural network.

4. The method of claim 2, wherein the segmentation model is configured to generate the segmented images as two-dimensional (2D) segmented images.

5. The method of claim 2, wherein the segmentation model is configured to generate segmented three-dimensional (3D) images of the vessel tree from the angiography images.

6. The method of claim 1, wherein the contrast intensity model is configured to determine a pixel metric for each segmented image and to determine a rising trend and a falling trend of the pixel metric over the sampling time window for the segmented images, the contrast intensity profile comprising the rising trend and the falling trend.

7. The method of claim 6, wherein the contrast intensity model is a machine learning model.

8. The method of claim 1, wherein the angiography images comprise images of the vessel tree in both (i) a baseline state and (ii) a hyperemic state.

9. The method of claim 8, further comprising determining, by the contrast intensity model, contrast intensity profiles for (i) angiography images of the vessel tree in the baseline state, and (ii) angiography images of the vessel tree in the hyperemic state, and wherein determining the microvasculature health comprises determining at least one of microvasculature resistance reserve or coronary flow reserve from the contrast intensity profiles of the baseline and hyperemic states.

10. The method of claim 1, the method further comprising:
obtaining additional angiography images of the vessel tree captured over a second sampling time window during which the contrast agent has been injected into the vessel tree, the additional angiography images captured at a different angle than the angiography images;
providing the additional angiography images to the segmentation model to generate a second set of segmented images of the vessel tree;
providing the second set of segmented images to the contrast intensity model and, performing, by the contrast intensity model, the contrast intensity extraction on each of the second set of segmented images and generating a second contrast intensity profile of the vessel tree over the second sampling time window; and
providing the second contrast intensity profile to the microvasculature health model.

11. The method of claim 1, the method further comprising:
obtaining additional angiography images of the vessel tree captured over a second sampling time window during which the contrast agent has been injected into the vessel tree, the additional angiography images captured at a different angle than the angiography images,
providing the additional angiography images to the segmentation model to generate a second set of segmented images of the vessel tree;
providing the second set of segmented images to the contrast intensity model and, performing, by the contrast intensity model, the contrast intensity extraction on each of the second set of segmented images and generating a second contrast intensity profile of the vessel tree over the second sampling time window;
comparing the contrast intensity profile and the second contrast intensity profile to reference contrast intensity profiles to identify one of the contrast intensity profile and the second contrast intensity profile as having a greater correlation to the reference contrast intensity profiles; and
providing the one of the identified contrast intensity profile or second contrast intensity profile having the greater correlation to the microvasculature health model.

12. The method of claim 1, wherein the sampling time window extends from an initial injection of the contrast agent into the vessel tree through washout of the contrast agent from the vessel tree.

13. The method of claim 1, wherein the microvasculature health model comprises a trained machine learning algorithm trained on training angiography images and at least one of (i) index of micro-circulatory resistance data, (ii) coronary flow reserve data, and (iii) microvascular resistance reserve data corresponding to the training angiography images, multi-physics simulation data corresponding to the training angiography images, and contrast intensity data.

14. The method of claim 13, wherein the microvasculature health model is trained to generate at least one of an index of micro-circulatory resistance, a coronary flow reserve value, and a microvascular resistance reserve value for the vessel tree.

15. The method of claim 13, wherein the microvasculature health model comprises an encoder stage for receiving the contrast intensity profile and a multilayer perceptron stage fed by the encoder and trained to generate at least one of a predicted index of microcirculatory resistance, a predicted coronary flow reserve value, and a predicted microvascular resistance reserve value as an indicator of the microvasculature health of the vessel tree.

16. A computer-implemented method for training a microvasculature health determination system, the method comprising:
obtaining angiography images of a plurality of vessel inspection regions from different subjects, the angiography images including subsets of angiography images captured over a full contrast agent injection cycle through corresponding vessel inspection regions, the angiography images include subsets of angiography images captured at different perspective views of corresponding vessel inspection regions;
obtaining vasculature health data for each of the angiography images;
performing a segmentation on each of the angiography images to generate a segmented image for each angiography image;
providing the segmented images to a contrast intensity model configured perform a contrast intensity extraction on each of the segmented images to generate a contrast intensity profile of a vessel tree in the vessel inspection region over a sampling time window; and
providing the contrast intensity profile and the vasculature health data to a machine learning model to train the machine learning model to generate a microvasculature health of a vessel tree in a subsequently imaged vessel inspection region.

17. The method of claim 16, wherein the vasculature health data comprises at least one of index of micro-circulatory resistance data, fractional flow reserve data, coronary flow reserve data, and microvascular resistance reserve data.

18. The method of claim 17, the method further comprising:
generating angiography images using a multi-physics model of a contrast injection through a vessel tree, the vessel tree having at least one coronary vessel branching into microvasculature vessels, the generated angiography images being binarized, and the generated angiography images corresponding to scenarios (i) baseline health microcirculation with baseline values of microvascular resistance, (ii) moderate disease microcirculation with moderate values of microvascular resistance, and (iii) severe disease microcirculation with high levels of microvascular resistance;
providing the generated angiography images to the contrast intensity model for generating contrast intensity profiles; and
providing the generated contrast intensity profile to the machine learning model to train the machine learning model to generate the microvasculature health of the vessel tree in the subsequently imaged vessel inspection region.

19. A method of assessing microvasculature function of a vessel inspection region for predicting a treatment response, the method comprising:
obtaining angiography images of a vessel tree in the vessel inspection region, the angiography images captured over a sampling time window during which a contrast agent has been injected into the vessel tree, and applying the angiography images to a segmentation model configured to generate segmented images of the vessel tree;
providing the segmented images to a contrast intensity model and, performing, by the contrast intensity model, a contrast intensity extraction on each of the segmented images to generate a contrast intensity profile of the vessel tree over the sampling time window;
providing at least a portion of the contrast intensity profile to a microvasculature health model configured to predict a response of the vessel inspection region to a treatment based on a characteristic of the at least a portion of the contrast intensity profile; and
generate an electronic indication of the predicted response to the treatment.

20. The method of claim 19, wherein the at least a portion of the contrast intensity profile is a downslope of the contrast intensity profile.

21. The method of claim 19, wherein the at least a portion of the contrast intensity profile is a portion of the contrast intensity profile isolated from an upslope portion of the contrast intensity profile.

22. The method of claim 19, wherein the microvasculature health model is configured to predict the response of the vessel inspection region to the treatment based on a downslope of the contrast intensity profile.

23. The method of claim 19, wherein the treatment is a medical procedure selected from the group consisting of a bypass procedure, coronary microvascular intervention, mechanical or ultrasound-based thrombectomy, angiogenesis therapy, stenting, or venous occluder.

24. The method of claim 19, wherein the treatment is a pharmacological treatment selected from the group consisting of an antiplatelet agent, an anticoagulant agent, a vasodilator agent, anti-inflammatory agent, a statin, nitroglycerin, calcium channel blockers, beta-blockers, ACE inhibitors, or other anti-anginal medications.
